(19) United States
(12) Patent Application Publication
(10) Pub. No.: US 2011/0104121 A1
(43) Pub. Date: May 5, 2011

(54) Female Reproductive Tract and Anal Prophylaxes

(76) Inventors: Charles R. Wira, Plainfield, NH (US); John V. Fahey, Enfield, NH (US); Charles L. Sentman, West Lebanon, NH (US); Paul M. Guyre, Lyme, NH (US)

(21) Appl. No.: 12/993,349

(22) PCT Filed: May 21, 2009
(86) PCT No.: PCT/US09/44767

§ 371 Date: Jan. 6, 2011

Related U.S. Application Data
(60) Provisional application No. 61/054,898, filed on May 21, 2008.

(51) Int. Cl. A61K 35/74; A61P 31/18; A61K 31/4709; C12N 5/00; A61K 31/573; A61K 31/4439; A61K 31/525; A61K 31/138; A61K 31/4535; A61K 31/55; A61K 31/40; A61K 31/519

(52) U.S. Cl. 424/93.2; 514/314; 435/375; 514/179; 514/338; 514/249; 514/651; 514/324; 514/217.08; 514/428; 514/259.31

(57) ABSTRACT

The present invention provides methods for boosting mucosal immunity in the female reproductive tract of pre- and post-menopausal women using a TGF-beta inhibitor, a Selective Estrogen Receptor Modulator, and/or a recombinant commensal bacterium that expresses endogenous microbicides into the intestinal tract or reproductive tract of a subject. It also provides methods for boosting innate and adaptive immunity by providing a glucocorticoid. Methods for preventing sexually transmitted infections including HIV infection are also provided.

### INTRODUCTION

This application claims benefit of priority from U.S. Provisional Patent Application Ser. No. 61/054,898, filed May 21, 2008, the content of which is incorporated herein by reference in its entirety.

This invention was made in the course of research sponsored by the National Institutes of Health, grant numbers AI-71761, AI-51877 and AI-13541. The U.S. government has certain rights in this invention.

### BACKGROUND OF THE INVENTION

The primary route of HIV infection throughout the world is via heterosexual contact. Once HIV has established infection, the virus rapidly mutates, and viral variants are selected and expand in vivo. Consequently, an effective immune response against established HIV infections has been difficult to induce. Innate immune defenses are the initial barriers that prevent the establishment of HIV and other infections.

Although not widely appreciated, it is possible for HIV as well as other sexually transmitted infections (STI) to infect cells within both the upper and lower female reproductive tract (FRT). In this regard, expression of HIV receptors and co-receptors by epithelial cells in the uterus has been demonstrated (Yeaman, et al. (2003) Immunology 109(1):137-46), wherein these cells express several receptors critical for HIV infection including CD4, CXCR4, CCR5 and galactosylceramide (GalC). Moreover, the expression of these receptors varies during the menstrual cycle suggesting a hormonal regulation of a woman's susceptibility to HIV infection. Furthermore, phenotypical analyses and infectivity studies of primary FRT cells identified potential targets of infection within the FRT (Howell, et al. (2005) Curr. HIV/AIDS Rep. 2(1):35-8).

Approaches to combat HIV infection of the FRT have been suggested. For example, WO 2005/115469 discloses a method for treating an IFN-gamma-mediated condition or disease such as HIV/AIDS in a mammal by administering to the mammal a Selective Estrogen Receptor Modulator (SERM) in an amount sufficient to reduce IFN-gamma expression or secretion, at least to some extent, in one or more lymphoid cells of the subject.

WO 2005/010049 teaches an isolated, and/or recombinant binding composition which specifically and/or selectively binds and neutralizes or antagonizes TGF-beta 1 for use in ameliorating, treating, preventing, and/or modulating a disease, disorder, syndrome, or condition of the immune system including HIV infection and HTLV-BLV infection.

WO 2007/012977 teaches a steroid kit including glucocorticoids such as cortisol and dexamethasone for use in treating, alleviating or preventing vaginal disorders, vulvar disorders, chlamydia infection, gonorrhea infection, hepatitis B, herpes, HIV/AIDS, and the like.

Furthermore, Liu, et al. (2006) Antimicrob. Agents & Chemother. 50:3250-3259) teach engineered vaginal Lactobacillus for mucosal delivery of the HIV inhibitor Cyanovirin-N. It was demonstrated that Lactobacillus-derived CV-N was capable of inhibiting CCR5-tropic HIV infectivity in vitro with a 50% inhibitory concentration of 0.3 nM. Moreover, the bacterial strain was capable of colonizing the vagina and producing full-length CV-N when administered intravaginally in mice during estrus phase.

Similarly, Natraj (2004) Current Science 86:1591-1592) teaches the use of L. jensenii, genetically engineered to produce and secrete CD4, to reduce the rate of HIV infection in cells. Further, co-incubation of the engineered bacteria with recombinant HIV-1_{HxB2} reporter virus led to a significant decrease in virus infectivity of HeLa cells expressing CD4-CXCR4-CCR5.

### SUMMARY OF THE INVENTION

The present invention embraces methods for boosting innate immunity in women, in particular pre-menopausal and post-menopausal. These methods involve contacting cells of the female reproductive tract with a TGF-beta inhibitor or a Selective Estrogen Receptor Modulator thereby boosting innate immunity. The present invention also relates to administration of low-dose glucocorticoid to enhance innate or adaptive immunity. Use of these same agents in methods for preventing sexually transmitted infections is also provided.

The present invention also embraces a method for preventing a sexually transmitted infection by administering to the intestinal tract or reproductive tract of a subject in need of treatment a recombinant commensal bacterium which expresses an endogenous microbicide. In some embodiments of this method, the bacterium selectively expresses the microbicide at least during mid-menstrual cycle, e.g., by being selectively expressed under the control of a hormone-responsive element.

Local delivery of the agents disclosed herein reduces or eliminates systemic effects of these inhibitors. Furthermore, it is possible to use inhibitors that only retain their biological effects within the local FRT tissues.

### BRIEF DESCRIPTION OF THE DRAWINGS

FIG. 1 depicts levels of MIP3 alpha secretion by primary uterine epithelial cells at 20 hours after exposure to estrogen, ICI 182,270 or the SERMs indicated. P value is comparison to media control.

FIG. 2 shows that Elafin can inhibit HIV-1×4/IIIB and R5 Bal. Recombinant Elafin at the amounts indicated was preincubated with virus for 1 hour at 37° C. The virus and Elafin solution was then added to TZM cells and inhibition of HIV-1 transcytosis was determined and expressed as a percent of control, i.e., no elafin.

### DETAILED DESCRIPTION OF THE INVENTION

HIV has multiple portals of entry through both the lower and upper FRT. Once deposited in the vagina, HIV, like sperm and radio-opaque dyes, moves rapidly from the lower FRT (vagina and ectocervix) into the upper FRT (endocervix, uterus, and Fallopian tubes). Immune cells in the FRT are phenotypically and functionally distinct from blood cells in that they are hormonally controlled to support sperm migration, fertilization and implantation. It has been shown that to support procreation, estradiol (E₂) acts directly, as well as indirectly through cytokines and chemokines and growth factors, to enhance or suppress essential components of the humoral, cell-mediated and innate immune systems in the upper and lower FRT (Wira, et al. (2005) Immunol. Rev. 206:306-35).

Menstrual cycle-dependent variations in populations of immune-relevant cells and molecules in the female reproductive tract increases susceptibility to infection by pathogens. The present invention now provides for the modulation of these endogenous populations of cells through sex hormone (e.g., estradiol/progesterone) modifiers, compensation through endogenous or exogenous antigens or engineered hormone-responsive microbes, or direct stimulation, addition, or suppression of specific immune-relevant cells and molecules thereby reducing or eliminating this susceptibility and specifically reducing the rate of infections that are transmitted via the FRT. Specifically, the present invention relates to the appreciation that Selective Estrogen Receptor Modulators (SERMs), TGF-beta inhibitors, glucocorticoids and engineered Lactobacillus can be used to optimize endogenous immune protection throughout the FRT thereby providing sustained protection throughout the menstrual cycle, and reversing the midcycle hormone-dependent suppression of immune function that places women at risk for infection by pathogens such as HIV-1 (e.g., X4 and R5).

The key observations which serve as a basis for this invention are as follows. Immune cells in the FRT are phenotypically and functionally distinct from blood and both the upper FRT and lower FRT provide immune defenses against potential pathogens (e.g., bacterial, fungal, and viral pathogens). Indeed, epithelial cells from throughout the FRT constitutively secrete antimicrobials, cytokines and chemokines that can be upregulated by TLR agonists. In this regard, it has been demonstrated that human uterine epithelial cell secretions inhibit the growth of pathogenic organisms such as Candida albicans and Neisseria gonorrhoeae, but not the commensal Lactobacillus crispatus.

One factor involved in susceptibility to pathogen infection in the FRT is TGF-beta. Specifically, it has been observed that immune cells, including epithelial cells in the FRT, produce TGF-beta that is biologically active. In particular, TGF-beta has been found to inhibit uterine natural killer (NK) cell responses to cytokines in human endometrium and has been further shown to regulate expression of HIV-1 receptors on myeloid dendritic cells.

Furthermore, IL-1β-mediated proinflammatory responses are inhibited by estradiol via down-regulation of IL-1 Receptor type I. Indeed, estradiol inhibits constitutive and TLR agonist-induced cytokine/chemokine secretion by uterine epithelial cells and stimulates secretory leukocyte protease inhibitor (SLPI) secretion by human reproductive tract cells, wherein said secretion in culture varies with menstrual status. Specifically, cytokines, chemokines and antimicrobials in uterine and cervical vaginal lavage specimens vary with stage of the menstrual cycle and are lowest at midcycle when estradiol levels are elevated. For example, MIP3α levels in uterine lavage drop from approximately 12 pg/uterine lavage in diestrus to approximately 9 pg/uterine lavage in proestrus and 4 pg/uterine lavage in estrus in the mouse uterus. Mouse KC (IL8) levels in mouse uterine lavage change from approximately 20 to 130 pg/uterine lavage when transitioning from diestrus to proestrus and drop to 30 pg/uterine lavage in estrus. In vivo secretion of MIP3α from vaginal lavage fluid follow a similar trend; ˜6.5 pg/50 μL vaginal lavage sample in diestrus, ˜0.6 pg/50 μL vaginal lavage sample in proestrus, and ˜1 pg/50 μL vaginal lavage sample in estrus.

Furthermore, estradiol has been shown to induce the mRNA expression of defensins by epithelial cells of the ectocervix, uterus, and Fallopian tube and decrease transepithelial resistance and NFκB expression. In addition, whereas sex hormones stimulate the accumulation of antimicrobials in uterine epithelial cell secretions, it inhibits these in cervical secretions. Moreover, whereas stromal fibroblasts from the Fallopian tubes, uterus and cervix produce hepatocyte growth factor (HGF), a known growth factor that mediates some estrogen effects on immune responses, only uterine HGF production increases with estradiol.

With regard to HIV-1, this hormone-dependent regulation of proinflammatory responses has significant implications. For example, primary uterine and Fallopian tube epithelial cell secretions both inhibit X4/T- and R5/M-Tropic HIV-1 replication and cervical vaginal lavage from non-HIV+ and HIV+ women and has been shown to contain a spectrum of antimicrobials that inhibit X4/T- and R5/M-Tropic HIV-1 replication. Moreover, epithelial cell secretions have been shown to reduce dendritic cell (DC) susceptibility to HIV-1 infection. Indeed, FRT NK cells, macrophages, dendritic cells, neutrophils, epithelial cells and fibroblasts produce soluble factors (SDF-1, RANTES, etc.) as well as antimicrobials (e.g., Defensins, SLPI, MIP3a, Elafin, etc) that can inhibit HIV infection, wherein these factors are regulated by sex hormones.

Endogenously produced microbicides in the secretions of the FRT have broad spectrum antimicrobial activity. Whereas conventional microbicide clinical trials focus exclusively on synthetic compounds, pH, etc. to interfere with the sexual transmission of HIV, the present invention embraces the use of endogenous peptides in the FRT as potent antimicrobials. By way of illustration, primary polarized epithelial cells from the Fallopian tubes, uterus, endocervix and ectocervix in culture were analyzed and shown to produce apical secretions capable of inhibiting bacterial, fungal and viral pathogens that compromise and threaten women's health. When apical secretions from polarized uterine epithelial cells recovered from premenopausal and postmenopausal women were incubated with STI for 1-2 hours prior to analysis of infectivity, epithelial secretions were effective in inhibiting Neisseria gonorrhoeae, Candida albicans and HIV-1, the causative agent of AIDS. Moreover, it was observed that secretions from epithelial cells of the Fallopian tubes, cervix and ectocervix also contained anti-microbial agents capable of inhibiting STI. A further unexpected finding from these studies was that FRT secretions had no effect on Lactobacillus crispatus, a commensal bacterium found in the lower FRT. These data indicate that anti-microbial agents in the FRT will not be lethal to the engineered commensal bacteria disclosed herein. These findings further provide definitive evidence for the critical role of upper and lower FRT mucosal epithelial cells in innate immunity to STI, particularly in controlling viral infectivity.

Data provided herein indicates that one or more endogenously produced microbicides or protease inhibitors in FRT secretions will provide effective protection against HIV and other STI. For example, partial inhibition of viral infectivity was achieved with purified anti-microbial agents, which, in some cases, was less than that seen with secretions from the FRT. Indeed, several microbicides made in the FRT exhibit anti-HIV activity. Specifically, MIP3 alpha and elafin have been shown to inhibit both X4 and R5 trophic virus. This was unexpected because prior to this analysis, these molecules were known exclusively for their antibacterial effects. Moreover, secretions from the FRT differently inhibit viral infection. Specifically, some viruses were more susceptible to FRT secretions than others, and while some CVL inhibit virus, others have no antiviral activity. These results indicate that antimicrobial agents in CVL vary from person to person, possibly as a result of hormone changes during the menstrual cycle.

Moreover, using a well-differentiated uterine epithelial cell line (ECC-1) and primary uterine epithelial cells (UEC), it has now been shown that conditioned medium from UEC selectively enhances expression of CCR5 and CXCR4 and concomitantly suppresses DC-SIGN expression on Dendritic cells during in vitro differentiation. Specific blockade of TGF-beta signaling with a pharmacological inhibitor of TGF-beta 1 receptor (SB431542) abolished these effects. In contrast, 17-β estradiol increases DC-SIGN expression on immature DCs. As such, uterine epithelial cell-derived TGF-beta may be a critical mediator for the regulation of susceptibility of DC infection in human endometrium.

It has also been observed that microbicides produced by immune cells in the upper FRT (Fallopian tubes, uterus and endocervix) contribute to downstream protection in the lower FRT (ectocervix and vagina). This is based on the findings presented herein that many of the molecules produced by uterine epithelial cells secrete the same molecules found in cervical vaginal washes.

The immune cells in the FRT contain a full spectrum of antiviral genes and their recognition molecules that are stimulated by TLR ligands. This represents an unanticipated level of intracellular protection that has not previously been demonstrated in the FRT. For example, it has now been found that the expression of MICA, an NKG2D ligand, in human endometrial cells is regulated by estradiol. Real-time PCR analysis showed that NKG2D ligands MICA and MICB were expressed in the human endometrium. MICA protein was detected primarily on epithelial cells, and greater expression was observed in immunohistochemical analysis of tissues from patients in the secretory phase of the menstrual cycle. Thus, these data suggest hormonal regulation of innate immunity and NKG2D-mediated recognition in other tissues and diseases where estrogen may be involved.

Accordingly, optimizing endogenous immune protection via regulation of hormone levels, down-stream effector molecules, and endogenous immune responses is embraced by this invention. Specifically, this invention provides the use of SERMs, TGF-beta inhibitors, glucocorticoids and engineered commensal bacteria to boost innate immunity throughout the FRT, in pre- and post-menopausal women. In addition, the present invention embraces the use of commensal bacteria expressing endogenous microbicides in anal prophylaxis of STIs such as HIV. In accordance with the present invention, an innate immune response or innate immunity is defined as the cells and mechanisms that provide immediate defense against infection by other organisms, in a non-specific manner. This means that the cells of the innate system recognize, and respond to, pathogens in a generic way, but unlike the adaptive immune system, it does not confer long-lasting or protective immunity to the host. As indicated, innate immune responses have been shown to be depressed during mid-menstrual cycle because of elevated estradiol levels. Agents of the present invention are described as boosting the innate immune response in so far as they reverse, alleviate, or enhance immune responses to elevated estradiol levels during mid-menstrual cycle thereby reversing, alleviating, or preventing the decrease in innate immune responses to pathogens which naturally occurs during mid-menstrual cycle.

On average, a menstrual cycle is 28 days long, but it can range from 21 to 35 days. By convention, the onset of menstrual bleeding (menstruation or menses) marks the beginning of the cycle. Menstruation lasts for several days and at about the middle of the cycle (i.e., mid-menstrual cycle, approximately 14 days before beginning of the next menstrual bleeding), estradiol levels are at the highest level when ovulation occurs. Thus, in accordance with the present invention, mid-menstrual cycle is defined as the time frame in which estradiol blood levels are at highest levels during the menstrual cycle, e.g., 1-3 days before ovulation and 7-10 days following ovulation.

Cells of the reproductive tract which are targets of the agents of the present invention include cells of the lower FRT (e.g., vagina and ectocervix) and upper FRT (e.g., endocervix, uterus, and Fallopian tubes). Such cells include, but are not limited to NK cells, macrophages, dendritic cells, neutrophils, epithelial cells and fibroblasts.

TGF-beta is a 24 kDa protein produced by many cells, including B and T lymphocytes and activated macrophages, as well as by many other cell types. Among the effects of TGF-beta on the immune system are inhibitions of IL-2-receptor induction, IL-1-induced thymocyte proliferation and blocking of gamma interferon-induced macrophage activation.

The effects of TGF-beta are mediated by the binding of active TGF-beta to specific receptors present on cells, followed by transduction of signal to those cells. TGF-beta inhibitors or antagonists are defined herein as molecules that inhibit TGF-beta signal transduction. TGF-beta antagonists are known in the art. For examples, molecules that bind TGF-beta and prevent TGF-beta from binding to a TGF-beta receptor will act as TGF-beta antagonists. Such molecules include neutralizing antibodies to TGF-beta, such as those described by Dasch, et al. ((1989) J. Immunol. 142:1536-1541), Lucas, et al. ((1990) J. Immunol. 145:1415-1422) and WO 2005/010049. Those skilled in the art recognize various ways in which an antibody derived from one species, for example a mouse, can be engineered in order to be therapeutically useful in a second species, for example a human.

TGF-beta is generally secreted as latent precursor consisting of TGF-beta non-covalently associated with a protein designated latency-associated protein (LAP; reviewed in Harpel, et al. (1992) Prog. Growth Factor Res. 4:321). This latent complex requires enzymatic cleavage of carbohydrate groups or transient acidification to release the active cytokine. Purified LAP by itself binds active TGF-beta with high affinity to form a latent complex. A DNA encoding a 278 amino acid peptide corresponding to pre-pro-TGF-beta, terminating just prior to the mature form of TGF-beta and containing a Cys33 to Ser33 substitution has been expressed (Derynck, et al. (1985) Nature 316:701), and found to bind TGF-beta and render it latent.

Soluble forms of TGF-beta receptors will also bind TGF-beta and prevent binding to membrane-associated TGF-beta receptors. TGF-beta receptors are described by Wang, et al. ((1991) Cell 67:797-805) and Lin, et al. ((1992) Cell 68:775-785). Soluble forms of TGF-beta receptors can be prepared by methods that are known in the art. For example, deletion mutants lacking the transmembrane domain of a TGF-beta receptor can be prepared, which will express a soluble TGF-beta binding protein. See, e.g., Miyazono, et al. (1994) Adv. Immunol. 55:181). Similarly, selective TGF-beta receptor inhibitors such as SB431542, LY364947, SD-208, and A-83-01 can be employed.

Other types of TGF-beta antagonists are also known in the art. For example, Yamaguchi, et al. ((1990) Nature 346:281-284) describe decorin as a small chondroitin-dermatan sulphate proteoglycan that binds TGF-beta and modulates the activity of this growth factor. Ohtsuki & Massague ((1992) Mol. Cell. Biol. 12:261-265) disclose protein kinase inhibitors that block certain biological activities of TGF-beta. See also Tsuchida, et al. (2006) Mini-Reviews Med. Chem. 6:1255; Saunier & Akhurst. (2006) Curr. Can. Drug Targets 6:565; Eriksson, et al. (2006) Am. J. Reprod. Imm. 56:321; Meadows, et al. (2006) Int. Immunopharm. 6:1020; Eriksson, et al. (2006) J. Immunol. 176:6219; Sato, et al. (2000) J. Immunol. 164:2285; Garba, et al. (2002) J. Immunol. 168:2247; Mselle, et al. (2007) Clin. Immunol. 124:69; Wira, et al. (2005) Immunol. Rev. 206:306.

Thus, TGF-beta antagonists of use in accordance with the present invention include, but are not limited to, blocking (neutralizing) antibodies specific for a human TGF-beta, soluble TGF-beta receptors, membrane-bound TGF-beta receptors, protease inhibitors that inactivate a protease responsible for activating a precursor TGF-beta into mature TGF-beta, antibodies specific to TGF-beta receptors (Types I, II or III) that prevent TGF-beta binding to the receptor, siRNA or antisense RNA that block expression of TGF-beta or combinations thereof. The activity and/or efficacy of TGF-beta inhibitors can be determined using the analysis disclosed herein or any suitable method routinely employed in the art. For example, TGF-beta expression can be determined by conventional northern or western blot analyses and phenotypic analysis can be employed to monitor uterine natural killer (NK) cell responses to cytokines in human endometrium or regulation of HIV-1 receptor expression on myeloid dendritic cells.

SERMs are a class of compounds that act on the estrogen receptor (Riggs & Hartmann (2003) N. Engl. J. Med. 348(7):618-29). SERMs of particular use in accordance with the present invention are mixed agonists/antagonists and pure antagonists of the estrogen receptor. Exemplary SERMs include, but are not limited to, triphenylalkylenes such as triphenylethylenes, which include Tamoxifen, Droloxifene, Toremifene, Idoxifene, Clomiphene, Enclomiphene and Zuclomiphene; benzothiphene derivatives such as Raloxifene, Y134 and LY 353381; benzopyran derivatives such as EM 800 (SCH 57050) and its metabolite EM 652; naphthalene derivatives such as Lasofoxifene (CP 336,156); chromans such as Levormeloxifene; bazedoxifene acetate and PHTPP; as well as analogs, derivatives, isomers, metabolites or mixtures of any of the above. In addition to SERMs, other ER antagonists such as ICI 182,780 are expected to useful in carrying out the methods of the present invention. The activity and/or efficacy of SERMs can be determined using the analysis disclosed herein or any suitable method routinely employed in the art.

Glucocorticoids are naturally-produced steroid hormones or synthetic compounds, which, when used at high levels suppress immunity and inflammation. However, antecedent or low-dose cortisol has now been found to enhance immune activation in response to TLR stimulation and enhance vaccine efficacy. Thus, the present invention provides for the use of antecedent or low-dose glucocorticoids to enhance immune activation. Low-dose glucocorticoid is defined herein as an amount of glucocorticoid which enhances adaptive or innate immunity in response to a pathogen. As exemplified herein, a 1.5 μg/kg/minute dose of hydrocortisone at 10 ml/hr for six hours provided a significant enhancement in the release of early pro-inflammatory molecules. As such, doses in the range of 0.5 to 5 μg/kg/minute are expected to be efficacious when administered as exemplified herein. Glucocorticoids of the invention can be either naturally-produced steroid hormones or synthetic compounds. Exemplary glucocorticoids for use in this invention include, but are not limited to, prednisone, prednisolone, methylprednisolone, dexamethasone, cortisol, hydrocortisone or combinations thereof. It is further contemplated that selective glucocorticoid receptor agonists can be employed in the instant method. The activity, efficacy, and determination of suitable dosing of a glucocorticoid can be determined based upon physiological studies in well-known animal models, wherein an enhancement of immune activation is the desired outcome.

In addition to the use of a TGF-beta inhibitor, a SERM or a glucocorticoid, particular embodiments of this invention embrace the use of an isolated and genetically engineered or recombinant commensal bacterium in the prevention and/or treatment of a sexually transmitted infection such as HIV. As is conventional in the art, commensal bacteria or indigenous microbiota are the beneficial microorganisms in direct contact with the intestinal and vaginal mucosal surfaces. According to the present invention, commensal bacteria are genetically engineered using conventional recombinant DNA technology and vectors to express endogenous or exogenous microbicides. Microbicides are molecules that can be applied inside the vagina or rectum to protect against sexually transmitted infections (STIs) including HIV. For the purposes of the present invention, an endogenous microbicide is a compound that is naturally found or produced by humans. Such molecules include, but are not limited to SLPI, Elafin, HBD2, and MIP3a. On the other hand, exogenous microbicides are molecules which exhibit microbicidal activity, but are not naturally found or produced by humans. Such molecules can include, but are not limited to for example the C-peptide derived from HIV-1_{89.6} gp41 C-terminal heptad repeated sequence. In particular embodiments, the microbicide of the invention is endogenous.

Commensal bacteria of particular use in accordance with the present invention include Lactobacillus sp. such as L. jensenii, L. acidophilus, and L. crispatus, and Staphylococcus epidermidis, which are naturally found in the vagina; and Escherichia coli, Lactobacillus sp. (e.g., L. fermentum, L. acidophilus), Bifidobacterium bifidum, Neisseria sp. and Streptococcus gordonii, which are naturally found in the intestinal tract. In particular embodiments, the recombinant commensal bacterium is a Lactobacillus sp. or E. coli.

HIV infections among men who have sex with men (MSM) are dramatically higher than those in the female and men who do not have sex with men populations. Therefore, according to one embodiment of the invention, the recombinant commensal bacteria of the invention are generated for anal prophylaxis in the MSM population. In accordance with this embodiment, E. coli and/or a Lactobacillus sp. are genetically engineered using standard methods to secrete one or more endogenous microbicides. In certain embodiments the endogenous microbicide is a microbicide naturally produced in the female reproductive tract. In particular embodiments, the microbicide is Elafin.

In another embodiment, the present invention provides for a recombinant Lactobacillus which selectively expresses one or more microbicides during the menstrual cycle, particularly during mid-cycle. Lactobacillus species of particular use in accordance with this embodiment are those Lactobacillus species that naturally colonize the vagina or recombinantly altered strains of such bacteria. Examples of these bacteria specifically embraced by this invention include L. jensenii and L. crispatus.

Commensal bacteria are described as being recombinant in that they have been genetically altered or modified by the introduction of a heterologous nucleic acid or protein. Thus, for example, recombinant Lactobacillus cells express genes that are not found within the native (nonrecombinant) form of the Lactobacillus or express native genes that are otherwise abnormally expressed, under-expressed or not expressed at all.

A commensal bacterium of the invention is modified to selectively express a microbicide throughout the menstrual cycle with special emphasis during mid-menstrual cycle as well as in postmenopausal women. In some cases, an anti-HIV agent of the invention will be a receptor that HIV binds to infect the host. In this regard, the Lactobacillus can express on its surface the normal host receptor for the virus, e.g., CD4 (known under GENBANK Accession No. NP_{—}000607), or virus-binding fragments thereof (e.g., 2D-CD4) (see, e.g., Orloff, et al. (1993) J. Virol. 67:1461-1471), stable CD4 trimers formed via a trimeric motif (see, e.g., Yang, et al. (2002) J. Virol. 76:4634-4642), a dodecameric CD4-Ig fusion protein (see Arthos, et al. (2002) J. Biol. Chem. 277:11456-11464) or CD4 in fusion with a single chain variable region of the 17b monoclonal antibody (mAb) (Dey, et al. (2003) J. Virol. 77:2859-2865). Alternatively, the anti-HIV agent is an agent that inhibits HIV replication, viability, or entry or otherwise binds to HIV. For example, alpha-defensins (Zhang, et al. (2002) Science 298:995-1000); cyanovirin-N (Bolmstedt, et al. (2001) Mol. Pharmacol. 59:949-54); human mAb b12, recognizing the CD4-binding site of HIV-1 gp120 (Saphire, et al. (2001) Science 293:1155-1159) or other molecules such as HIV-1 entry inhibitory protein (e.g., Root, et al. (2001) Science 291:884-888; Sia, et al. (2002) Proc. Natl. Acad. Sci. USA 99:14664-14669). SLPI, Elafin, HBD2, and MIP3a are contemplated for use as anti-HIV agents.

In accordance with the present invention, a microbicide is selectively expressed during mid-menstrual cycle by placing the coding sequence of the microbicide under the control of a hormone-responsive element, e.g., one or more estrogen response elements or progesterone response elements, and coexpressing in the recombinant Lactobacillus the cognate receptor, e.g., an estrogen receptor or progesterone receptor, respectively. Upon an increase in estrogen or progesterone level, the recombinant Lactobacillus selectively responds by expressing and secreting the microbicide. Lactobacillus species can be genetically engineered to co-express a hormone receptor, and a microbicide under transcriptional control of a hormone response element using routine techniques in the field of recombinant biology. Likewise, recombinant expression of a microbicide for anal prophylaxis can be carried out using routine techniques. Basic texts disclosing the general methods of use in this invention include Sambrook, et al. (2001) Molecular Cloning, A Laboratory Manual (3rd ed.) and Kriegler (1990) Gene Transfer and Expression: A Laboratory Manual. A general discussion of recombinant techniques for expressing and secreting proteins in Lactobacillus species that naturally colonize the vagina is found in U.S. Patent Application No. 20070117197. The protein and nucleotide sequences for estrogen and progesterone receptors are well-known in the art. See, e.g., GENBANK Accession No. NP_{—}000116 (human estrogen receptor alpha), NP_{—}001035365 (human estrogen receptor beta), and NP_{—}000917 (human progesterone receptor). Similarly, estrogen and progesterone response elements are well-known and clearly described in U.S. Pat. Nos. 5,770,176 and 7,045,315, respectively.

Boosting innate immunity throughout the FRT, in particular during mid-menstrual cycle hormone-dependent suppression of immune responses, via administration of a TGF-beta inhibitor, a SERM, a glucocorticoid, a recombinant Lactobacillus, or combinations thereof is useful in the prevention of any sexually transmitted infection of the FRT including fungal (e.g., Candida albicans), viral (e.g., HSV, HIV), and bacterial (e.g., Escherichia coli, Gardnerella vaginalis, Mycoplasma bovis, Mycoplasma hominus, Neisseria gonorrhoeae, Staphylococcus saprophyticus, Chlamydia trachomatis) infections and, in particular embodiments, in the prevention of HIV infection. Likewise, administration of a recombinant commensal bacterium for anal prophylaxis in the MSM population is useful in the prevention of fungal, bacterial and viral infections. Prevention, in the context of HIV infection means a reduction or inhibition of entry of HIV into a cell and/or the lack of further increase in entry, which would occur in the absence of the inhibitor. Thus, an effective amount of an agent, Lactobacillus, or other commensal bacterium of the invention is an amount which achieves the desired result of blocking or inhibiting transmission or entry of HIV into or between cells.

The term “subject,” as used herein, refers to an animal, in general, a warm-blooded animal. Subjects specifically embraced by the present invention include cattle, buffalo, sheep, goats, pigs, horses, dogs, cats, rats, rabbits, mice, and humans. Also included are other livestock, domesticated animals and captive animals. In particular embodiments, the subject is human.

An agent, Lactobacillus, and/or other commensal bacterium of the invention can be administered for prophylactic and/or hygienic use. Administration is generally topical, particularly to epithelial surfaces of the intestinal tract or female reproductive tract, especially the mucosal surfaces. Administration of such agents and recombinant commensal bacteria in accordance with the present invention can be in a single dose, in multiple doses, in a continuous or intermittent manner, depending, for example, upon the recipient's physiological condition and other factors known to skilled practitioners. For prevention of infection, administration of the agents and recombinant commensal bacteria of the invention can be essentially continuous over an indeterminate period of time, for example, at regular intervals for life. Alternatively, agents and recombinant commensal bacteria of the invention can be administered continuously for a pre-selected period of time or in a series of spaced doses, e.g., only during mid-menstrual cycle. Moreover, administration can be manual or automatic, e.g., in response to changing hormone levels.

Agents and/or recombinant commensal bacteria are prepared by combining the active ingredient in the appropriate concentration. Other active or inactive agents selected by one of skill in the art can optionally be added. The absolute weight of a given active agent included in a unit dose can vary widely.

Agents and/or recombinant commensal bacteria of the invention can be administered to the intestinal or female reproductive tract in the form of an article or carrier such as an insert, syringe-like applicator, suppository, tablet, powder/talc or other solid, suspension, solution, emulsion, liquid, spray, aerosol, douche, ointment, tampon, foam, cream, gel, paste, microcapsules, time-release capsules, vaginal sponge, vaginal ring, controlled release formulation, sustained release formulation or bioadhesive gel (e.g., a mucoadhesive thermogelling composition as appropriate; see, for example, U.S. application Ser. No. 10/135,805).

For intravaginal administration, an agent and/or recombinant Lactobacillus can be formulated as is known in the art for direct application to the vaginal area. Forms chiefly conditioned for vaginal application include, for example, creams, milks, gels, dispersion or micro-emulsions, lotions thickened to a greater or lesser extent, impregnated pads, ointments, aerosol formulations (e.g., sprays or foams). Alternatively, the agent and/or recombinant Lactobacillus can be formulated to be part of an adhesive polymer, such as polyacrylate or acrylate/vinyl acetate copolymer.

Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents. Liquid sprays are conveniently delivered from pressurized packs, for example, via a specially shaped closure. The active compositions can also be delivered via iontophoresis, e.g., as disclosed in U.S. Pat. No. 4,140,122; 4,383,529; or 4,051,842. The percent by weight of a prophylactic agent of the invention present in a formulation will depend on various factors, but generally will be from about 0.01% to about 98% of the total weight of the formulation, and typically about 0.1 to about 90% by weight.

The pharmaceutical formulations of the present invention can include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions with a pH of about 4.0 to about 7.4.

Furthermore, the active ingredients can also be used in combination with other therapeutic agents, for example, anti-microbial agents, pain relievers, anti-inflammatory agents, vitamins (e.g., vitamin B, C or E), aloe vera and the like, whether for the conditions described or some other condition. Furthermore additional beneficial agents can be included that can improve the intestinal or vaginal environment. For example, polymers used as carrier or for encapsulation or for sustained-release can be hydrolytically degraded into an acid or acid producing species. One such polymer is a poly (vinyl alcohol) backbone with pendant polycaprolactone chains that, upon disintegration, yields poly (vinyl (polycaprolactate)). The polycaprolactone is hydrolytically degraded into caproic acid. This acid aids in lowering pH and controlling harmful bacterial growth, thus helping to restore balance to the vaginal system. Additionally, a peroxide of Laureth-4 (e.g., a Laureth-4 terminal peroxide) would release laureth-4 and peroxide (e.g., hydrogen peroxide). Laureth-4 decreases TSS-1 production by S. aureus and the peroxide is available to suppress undesirable anaerobes and Gardnerella vaginalis, thus reducing toxin production while reestablishing the vaginal flora.

As will be apparent to those skilled in the art, ingredients of the formulation can be varied to affect certain properties of the formulation. For example, the viscosity can be varied by adding a polymer or gel former.

In some embodiments, a bioadhesive polymer can be included at various concentrations to provide greater or lesser bioadhesion.

The invention is described in greater detail by the following non-limiting examples.

### Example 1

### Regulation of HIV-1 Receptor Expression on Myeloid Dendritic Cells Via TGF-Beta

Materials and Methods. Uterine epithelial cell line (ECC-1) was cultured according to established methods to establish cellular polarity with apical and basolateral compartments (Schaefer, et al. (2004) Immunology 112:428-436). The formation of tight junctions by the epithelial cell monolayer was routinely monitored by measurement of transepithelial resistance (TER) (Richardson, et al. (1995) Biol. Reprod. 53:488-498). Basolateral conditioned medium (CM) was collected from the ECC-1 cells following 24-hour culture in complete DMEM medium (Hyclone, Logan, Utah) supplemented with 20 mM HEPES, 50 U/ml penicillin, 50 mg/ml streptomycin, 2 mM 1-glutamine (all from Life Technologies) and 10% heat-inactivated defined fetal bovine serum (Hyclone, Logan, Utah) and did not contain phenol red. The pooled media was centrifuged for 5 minutes at 10,000 g and stored at −80° C. for use in subsequent studies.

Peripheral blood mononuclear cells (PBMC) were obtained from residues of platelet pheresis according to known methods (Dietz, et al. (2006) Transfusion 46:2083-2089). Monocytes were subsequently enriched with Rosette Sep reagent (StemCell, Vancouver, BC). Blood specimens were from healthy adult males. Monocytes-derived Dendritic cells (MDDCs) were in vitro generated with GM-CSF (50 ng/ml) and IL-4 (50 ng/ml) (PeproTech Inc, Rocky Hill, N.J.) (Sallusto Lanzavecchia (1994) J. Exp. Med. 179:1109-1118). MDDCs were differentiated for 7 days in the presence or absence of 24-hour conditioned medium (1:1 dilution) obtained from ECC-1. In some experiments TGF-beta receptor 1 blocker, SB431542 (10 μM; Tocris Cookson, Inc., St. Louis, Mo.) (Eriksson, et al. (2006) Am. J. Reprod. Immunol. 56:321-328; Shen, et al. (2007) Clin. Exp. Immunol. 149(1):155-61) was simultaneously added with ECC-1 CM for the entire duration of the differentiation. As positive control for TGF-beta effects on MDDCs, experimental conditions were included in which MDDCs were differentiated with recombinant human TGF-beta1 at three different concentrations (0.1, 1 and 10 ng/mL) in the presence or absence of SB431542. Following 7 days of culture, cells were harvested, washed and subsequently used for flow cytometry analyses.

Following six days of in vitro differentiation, surface staining of MDDCs was performed with the following fluorochrome-labeled monoclonal antibodies: CD1a (HI149), CD14 (61D3), from Ebioscience (San Diego, Calif.); and CD163 (215927), CCR5 (45531), CXCR4 (12G5), DC-SIGN (120507) from R&D Systems (Minneapolis, Minn.). Matched isotype controls for the antibodies were used to control for non-specific binding. Following antibody staining, cells were washed with staining buffer and fixed with 2% methanol-free paraformaldehyde in 1×PBS (2% PFA), followed by analysis with a Becton Dickinson FACS CALIBUR (San Jose, Calif.). The acquired FACS data were analyzed by CELL QUEST (BD Biosciences).

Concentration of TGF-beta in ECC-1 conditioned medium or in the supernatants from primary uterine epithelial cells was determined with the DUOSET ELISA development kit (R&D Systems) according to the manufacturer's recommendations. Mature TGF-beta was directly assayed in the samples, whereas total TGF-beta (latent and mature forms) was assayed after treatment with 0.1 M HCl at room temperature for 10 minutes followed by neutralization of sample with 1.2 M NaOH/0.5 M HEPES. The assay limit of detection is 31.1 pg/ml.

Statistical analysis was performed using two-way analysis of variance (ANOVA) (GRAPHPAD Prism Version 5.0; GRAPHPAD Software Inc., San Diego, Calif.). A value of P<0.05 was considered significant.

Results. To determine the effect of uterine epithelial cell secretions on the surface expression of DC-SIGN on MDDCs, highly human monocytes were differentiated in the presence (i.e., conditioned DCs, CM-DC) or absence (control DC, Con-DC) of ECC-1 CM for seven days. Cells were then analyzed for surface expression of DC-SIGN. The results of this analysis of DCs indicated that CM from ECC-1 cells had decreased expression of DC-SIGN as compared to Con-DC. Combined data from five different donors indicated that the presence of ECC-1 CM during differentiation of MDDCs significantly down-regulated the expression of DC-SIGN. To determine the effect of acute effect of ECC-1 CM on DC-SIGN expression by DCs, Con-DCs were cultured for an additional 48 hours in the presence of ECC-1 CM. This analysis indicated that short-term treatment of MDDCs with ECC-1 CM resulted in the down-regulation of DC-SIGN expression.

In addition to DC-SIGN, CCR5 and CXCR4, the effect of ECC-1 CM on the expression of CD1a, C14 and CD163 was also analyzed. This analysis indicated that the expression of CD1a was not significantly different on Con-DC and CM-DC. Neither was the expression of CD14 nor CD163 on MDDCs significantly affected by ECC-1 CM. These findings collectively indicate that ECC-1 CM selectively modulated the expression of DC-SIGN and the chemokine receptors.

TGF-beta is a cytokine with profound effects on DCs (Strobl & Knapp (1999) Microbes Infect. 1:1283-1290). It is highly expressed within human endometrium tissues (Godkin & Dore (1998) Rev. Reprod. 3:1-6). Further, uterine epithelial cells (UECs) produce TGF-beta (Shen, et al. (2007) supra; Kim, et al. (2005) Mol. Hum. Reprod. 11:801-808). It was therefore determined whether the effects of ECC-1 CM on expression of DC-SIGN and the chemokine receptors by MDDCs was dependent on TGF-beta. MDDCs were differentiated in the TGF-beta receptor inhibitor (SB431542). This analysis indicated that ECC-1 CM significantly suppressed the expression of DC-SIGN on MDDCs. However, in the presence of TGF-beta receptor inhibitor, this modulatory effect of ECC-1 CM was abrogated. TGF-beta receptor blocker had no significant effect on the expression of CD1a by CM-DC. These findings indicate that ECC-1 CM effect on the expression of DC-SIGN, CCR5 and CXCR4 by MDDCs is partly dependent on TGF-beta1.

To determine the production of TGF-beta by uterine epithelial cells, culture supernatants obtained from ECC-1 were assayed for latent and active TGF-beta. In this assay, only latent TGF-beta was detected. ECC-1 secreted TGF-beta1 into the apical and basolateral chambers in equal proportions. Similarly, TGF-beta production was demonstrated in the culture supernatants from primary UEC. These results indicate that UEC down-regulate expression of DC-SIGN and up-regulate expression of CCR5 and CXCR4 and that this effect is likely mediated by TGF-beta.

### Example 2

### Immunoprotective Effects of TGF-Beta Inhibitors

The present invention relates to the activation of innate immune defenses, such as intracellular antiviral proteins, secreted defensins, or soluble cytokines, within the mucosal tissues to prevent infection by a pathogen such as HIV-1. In one embodiment, TGF-beta inhibitors are administered locally and directly to the FRT. The delivery of the inhibitors locally reduces or eliminates systemic effects of these inhibitors. Furthermore, it is possible to use inhibitors that only retain their biological effects within the local FRT tissues. Such localized delivery of TGF-beta inhibitors is expected to modulate innate immune responses within these tissues and decrease HIV coreceptor expression to prevent HIV entry thereby leading to prevention of infection rather than treatment of an ongoing infection. TGF-beta has three isoforms, and these inhibit many functions of adaptive and innate immune cells. TGF-beta is widespread in the FRT and is produced as an inactive propeptide. The propeptide resides in tissues and remains inactive until cleavage of the peptide into its mature active form. TGF-beta binds to type I and type II serine/threonine kinase receptors, which mediate intracellular signaling. HIV induces TGF-beta production, which results in increased T cell and NK cell apoptosis. Moreover, TGF-beta induces expression of CCR5 and CXCR4, which are coreceptors for HIV entry into cells.

Within the FRT, a variety of innate immune cell types mediate effector responses that have the potential to inhibit STI including HIV infection. It has been found that TGF-beta inhibitors block endogenous TGF-beta, leading to increases in the effector responses of human uterine NK cells and total endometrial cells. TGF-beta blockade does not result in spontaneous innate cell activation, but results in greater effector responses only when microbial signals such as peptidoglycan were present.

The ability of TGF-beta inhibitors to alter key innate immune effector functions and promote factors that inhibit HIV infection is determined as follows. FRT tissues obtained from surgical specimens are used as a source of primary human cells. Tissues are obtained from women with a variety of clinical conditions and are distal to any pathology. Primary cells are isolated using a collagenase/DNAse enzyme digest. Both stromal cells (containing leukocytes) and epithelial sheets are isolated. Using conventional cell purification and/or multi-color flow cytometry techniques, a variety of effector responses and anti-HIV activity are evaluated in FRT tissues and cells (e.g., Fallopian tube, endometrium, cervix, ectocervix, vaginal mucosa) treated with TGF-beta inhibitors. TGF-beta inhibitors, SB431542, LY364947, SD-208, A-83-01, are prepared in an appropriate vehicle, and activity and cell toxicity of these compounds is analyzed on human FRT cells under dose response conditions.

Specifically, the following readouts are evaluated in the presence and absence of TGF-beta inhibitors to determine effector responses and anti-HIV activity.

The extent to which soluble factors from innate cells prevent HIV infection is determined using the TZM-bl assay and infection of PBMCs, e.g., as measured by p24 ELISA. Several HIV viral strains are tested, including clinical isolates, X4, and R5 viral strains.

Secretory leukocyte protease inhibitor (SLPI) and human beta-defensin 2 (HBD2) production by primary epithelial cells is evaluated and compared to molecules known to inhibit HIV.

Activation of intracellular anti-viral pathways (e.g., MxA, OAS, PKR) are monitored using conventional methods. (See, e.g., Unoshima, et al. (2003) Antimicrob. Agents Chemother. 7(9): 2914-2921).

Activation of local NK cell cytokine production which can inhibit HIV infection and activate innate immune defenses is evaluated. Specifically, activation of NK cell production including IFN-gamma and SDF-1alpha is determined.

Alterations in the expression of HIV coreceptors, CCR5 and CXCR4, on FRT cells is determined using conventional immunodetection methods.

Results of this analysis are expected to show that inhibition of TGF-beta signaling in primary human FRT cells leads to increases in effector molecules with known anti-HIV activity, a decrease in CCR5 and CXCR4 co-receptor expression, and an increased ability of FRT cell-conditioned medium to inhibit STI as well as HIV infection of cells.

### Example 3

### Estradiol Modulation of Hepatocyte Growth Factor

Materials and Methods. FRT mucosal tissue was obtained following surgery from women who underwent hysterectomies. Tissues used in this study were distal to the sites of pathology and were determined to be unaffected by disease upon inspection by a trained pathologist. Tissues were transported from Pathology and procedures to isolate stromal fibroblasts began within 2 hours of surgery.

Tissues from the Fallopian tube, uterus, cervix, and ectocervix were dispersed into epithelial and stromal cell fractions by enzymatic digestion (Fahey, et al. (1998) Immunol. Invest. 27:167-180; Meter, et al. (2005) Fertil. Steril. 84:191-201). Briefly, tissues were minced under sterile conditions into 1-2 mm fragments and then digested at 37° C. for 1 hour using a “PHC” enzyme mixture that contained final concentrations of 3.4 mg/ml pancreatin (Gibco Life Technologies, Rockville, Md.), 0.1 mg/ml hyaluronidase (Worthington Biochemical Corporation, Freehold, N.J.), 1.6 mg/ml collagenase (Worthington) and 2 mg/ml D-glucose (Sigma, St Louis, Mo.) in Hank's balanced salt solution (Gibco, Invitrogen). This procedure was chosen to maximize digestion of the extracellular matrix, as verified by microscopy and by hematoxylin and eosin staining of frozen sections after digestion, while minimizing digestion of cell surface antigens as determined by flow cytometry. After incubating in this enzyme cocktail, cells and tissue fragments were collected, washed with complete media consisting of DMEM/F-12 without phenol red (Gibco) supplemented with 10% heat-inactivated fetal calf serum (Hyclone, Logan, Utah), 2 mM fresh L-glutamine, 25 mM HEPES, 50 ng/ml primocin (InvivoGen, San Diego, Calif.) and separated from tissue aggregates by sedimentation for 5 minutes. Medium containing predominantly single cells and small epithelial cell glands were removed and dispersed through a 250 μm nylon mesh screen (Small Parts, Miami Lake, Fla.). To further separate epithelial and stromal cells, cells were resuspended in complete media, vortexed twice at low speed for 10 seconds, repeating the sedimentation and dispersal and dispersed cells were then pooled and separated on a 40 μm mesh screen (Small Parts) into fractions containing epithelial cell glands and a mixed population of sub-epithelium stromal cells. Stromal cells passed through this fine mesh whereas small epithelial cell glands did not.

Stromal cell fractions were washed, resuspended in complete media, and cultured in T75 flasks. Twenty-four hours after seeding, flasks were washed and agitated to remove remaining red blood cells and other non-fibroblastic stromal cells. When confluent, the cells in the flask were counted and sub-cultured into 24 well culture plates (Fisher Scientific, Pittsburgh, Pa.). After 4 days of culture, all cells had the characteristic fibroblast morphology, and the culture was devoid of other cell types. Media was replaced at 48-hour intervals as the fibroblasts grew to confluence.

Once the cells reached confluence, they were cultured in complete media with 10% stripped FBS (Hyclone). After 48 hours of acclimation to this media, cells were treated with 17β-estradiol (CalBiochem, San Diego, Calif.). Estradiol was dissolved in 100% ethanol, evaporated to dryness and resuspended in media. For control medium, an equivalent amount of ethanol was initially evaporated prior to the addition of media. Treatment groups consisted of four wells per treatment. Supernatants from each well were collected at 48-hour intervals, centrifuged at 10,000×g to remove any cellular debris, and stored at −80° C. until assayed. Cells in culture were resupplemented with fresh complete media and re-treated every 48 hours until the indicated time courses were complete. In related experiments, when cell proliferation was analyzed, harvested from culture wells and counted by Coulter Counter. Briefly, culture media was removed, 0.05% trypsin (Mediatech, Inc., Herndon, Va.) with 0.5 nm EDTA added for minutes, cells were recovered and washed with fresh media. Viability of the cultures was determined using trypan blue dye exclusion and manual counting of live vs. dead cells on a hemocytometer.

Culture supernatants were analyzed for HGF via a commercially available ELISA DUOSET (QUANTIKINE; R&D system, Minneapolis). Concentrations of HGF in the supernatants were measured in quadruplicate. The limit of sensitivity for this assay was 50 pg/ml.

The data for HGF secretion by the uterine, cervical, and ectocervical stromal fibroblasts are shown as mean±SEM. InSTAT Software was used to perform a one-way repeated-measures analysis of variance (ANOVA). When an ANOVA indicated that significant differences existed among means, pre-planned paired comparisons were made using the Dunnet method to adjust P-values. A P-value of <0.05 was considered statistically significant.

Results. Primary stromal fibroblasts were isolated and cultured from uterine, cervical and ectocervical tissues from hysterectomy patients. Tables 1 and 2 summarize patient age and 48 hour constitutive HGF production by FRT stromal fibroblasts cells.

TABLE 1

| | | Constitutive HGF Production |
|---|---|---|
| | | (pg/ml ± SEM) by Uterine |
| Patient Number | Age | Stromal Fibroblasts (48 hours) |
| #3126 | 48 | 815 ± 47 |
| #3138 | 46 | 365 ± 50 |
| #3677 | 39 | 485.2 ± 12 |
| #3678 | 47 | 550.3 ± 47 |
| #3729 | 50 | 146 ± 16 |
| #3821 | 50 | 181.9 ± 4 |
| #3851 | 50 | 351.3 ± 5 |
| #3917 | 65 | 158.7 ± 39 |
| #3976 | 46 | 2716.7 ± 102 |

TABLE 2

| | |
|---|---|
| | Constitutive HGF Production |
| | (pg/ml ± SEM) by Fibroblasts |
| Patient | (48 hours) |

| Number | Age | CX | ECX |
|---|---|---|---|
| #4247 | 54 | 3594 ± 190 | 983.1 ± 100 |
| #4263 | 44 | 1543.8 ± 155 | 2017 ± 343 |
| #4485 | 66 | 438 ± 86 | 467 ± 46 |

In all cases, cells were routinely grown to confluence (about 7 days) prior to the induction of treatment and/or HGF analysis. As shown in Table 1, constitutive HGF secretions by uterine stromal fibroblasts derived from nine patients varied from 140 pg/ml to 2716.7 pg/ml. The ages of the patients ranged from 39-65 years old. Patient variables such as age and reason for surgery indicated that hormonal effects transcended the reason for surgery and could not be correlated with HGF secretion. Since the tissues were excised distant from the sites of any pathology, it is unlikely that abnormal cells were obtained. Cells were assessed for HGF secretion beginning about 10 days following surgery and after repeated media changes at 48 hours. Table 2 shows 48 hour HGF secretion from three matched cervical (CX) and ectocervical (ECX) tissues. All three tissue sources exhibited HGF secretion by stromal fibroblasts in culture.

To evaluate the ability of primary uterine stromal fibroblasts to produce HGF constitutively, purified human uterine stromal cells were grown to confluence in 24-well plates, and CM collected and replenished at 48-hour intervals for 8 days. This analysis indicated that human uterine stromal fibroblasts produced HGF constitutively, with secretion continuing for at least 8 days; in uterine stromal fibroblasts from one patient, continued secretion was measured for 12 days. Consistent HGF secretion was seen in primary cultures of uterine stromal fibroblasts from 8 out of 8 donors used for time-course experiments.

To determine whether estradiol influences HGF production, confluent monolayers were incubated with media in the presence or absence of 10⁻⁷ M or 10⁻⁸ M estradiol for 48-hour intervals, at which time CM was collected, and analyzed for HGF by ELISA. This analysis indicated that estradiol significantly increased uterine stromal fibroblast HGF secretion at both treatment levels within 48 hours. In other experiments HGF was measured at 24 hours following the addition of estradiol. Under these conditions, an estradiol response was observed in two out of five patients. In all further experiments, the effects of estradiol were focused on the period of time following 48 hours incubation.

To determine whether HGF secretion increased with time of exposure to estradiol, cells were incubated with estradiol for increasing lengths of time. This analysis indicated that the estradiol-induced secretion of HGF from uterine stromal fibroblasts increased with the duration of exposure to estradiol. Uterine stromal fibroblasts were treated for 2, 4, and 6 days, and CM was collected at 48 hour intervals. Cells treated with estradiol for six days secreted three times more HGF than cells exposed for two days. The continued increase of HGF secretion induced by estradiol over 6 days was in contrast to the effects of estradiol on MIP3α secretion by rat stromal fibroblasts (Crane-Godreau & Wira (2004) Infect. Immun. 72:1866-187). In that study, the increase in secretion of MIP3α by estradiol was gone by 24 hours. This long-lasting effect of estradiol could have implications for cancer or endometriosis where continued HGF secretion could lead to further proliferation of uterine epithelial cells.

Since estradiol is known to affect cell proliferation (Gruber, et al. (2002) N. Engl. J. Med. 346:340-352), it was determined whether the estradiol-induced increase in HGF secretion by uterine stromal fibroblasts was due to increased secretion by existing cells or if stromal cell proliferation due to estradiol treatment resulted in higher HGF secretion. Cultured uterine stromal fibroblasts were harvested and counted at 48-hour intervals beginning at the start of estradiol treatment. For those cultures analyzed, media was replenished at intervals day 2, 4, and 6. This analysis indicated that cell numbers in the control and estradiol-treated wells did not differ, suggesting that HGF secretion was increased by estradiol treatment and not due to changes in cell numbers.

Moreover, in contrast to uterine stromal fibroblasts, estradiol did not increase HGF secretion by cervix stromal fibroblasts. Likewise, estradiol also had no effect on the secretion of HGF from ectocervix stromal fibroblasts.

### Example 4

### Immunoprotective Effects of SERMs

In women, E₂ plays a key role in reproduction and has beneficial effects on the skeletal, cardiovascular, and central nervous systems. Most estrogenic responses are mediated by the estrogen receptors ER alpha and ER beta, which are members of the nuclear receptor superfamily of ligand-dependent transcription factors. Selective ER modulators (SERMs) are a class of pharmacological compounds that are used to regulate ER-mediated responses in vivo. SERMs in clinical use include tamoxifen and raloxifene, which are administered for treatment of breast cancer and osteoporosis. SERMs have cell-type specific agonist or antagonist effects, depending on the cellular complement of coactivators or corepressors (Dutertre, et al. (2000) J. Pharm. Exp. Ther. 295:431).

All major aspects of the mucosal immune system throughout the FRT have been systematically examined and the cyclic effects of E₂ on their functions has been defined. Working with secretions, fresh tissues and isolated immune cells, the immune system has been mapped in terms of immune cell phenotype, function and hormone regulation during the menstrual cycle. It has been shown that immune cells, cell secretions, and cervical-vaginal lavage (CVL) samples from women are under hormonal control and are effective in inhibiting HIV-1 (×4 and R5). These findings provide direct evidence for the critical role of the upper and lower FRT in providing immune protection against HIV.

Accordingly, the present invention provides for the regulation of cyclic endogenous anti-microbial activity in the human FRT via SERMs or other ER antagonists. SERMs can be used to selectively sustain and enhance immune protection at a time when the FRT is susceptible to infection without compromising normal reproductive function or increasing the risk of HIV infection. It is expected that SERMs selectively stimulate uterine, cervical, ectocervical and vaginal immune cell protective responses against STI without enhancing those immune parameters that promote STI infection. This approach uses the endocrine system to optimize FRT endogenous immune protection that has evolved to protect against viral pathogens including HIV. Specifically, this approach focuses on boosting endogenously produced microbicides shown to have antifungal, antibacterial and antiviral activity. Moreover, in contrast to conventional approaches, it has now been appreciated that the upper FRT is both a potential site of HIV infection and a source of protective microbicides that are secreted throughout the upper and lower FRT. The use of SERMs in this approach is based on the demonstration that E₂ stimulates human uterine epithelial cell secretion of innate microbicides (defensins, SLPI, etc.) in vitro while suppressing chemokines (IL-8, etc.) that enhance the risk of infection; that these same antimicrobials in situ in CVL are suppressed at midcycle when E₂ levels are elevated; and secretions from isolated FRT cells and CVL inhibit X4 and R5 HIV infection of target cells.

The analysis of SERM activity is carried out using fresh hysterectomy tissues, wherein epithelial cells, neutrophils, NK cells and macrophage/dendritic cells (MAC/DC), which are known to be responsive in culture to sex hormones, are recovered. Using conventional assays, SERMs (e.g., Tamoxifen, bazedoxifene acetate, lasofoxifene and raloxifene, etc) are analyzed for their ability to regulate immunological endpoints which have been identified as optimal for protection against HIV. For example, it was observed ICI 182,780, tamoxifen, or PHTPP resulted in an increase in relative uterine SLPI mRNA levels of ˜3×10⁵, ˜2.5×10⁵, and ˜8×10⁵, respectively, over saline controls. Similarly, ICI 182,780 increased relative vaginal SLPI mRNA levels by 26-fold over saline controls. Furthermore, ICI 182,780, tamoxifen, and Y134 increased relative uterine alpha defensin 2 expression (˜3×10⁵, ˜4×10⁵, and −1.2×10⁶, respectively) compared to ˜0.2×10⁵ in the saline control. ICI 182,780, Y134, and PHTPP were also found to increase relative vaginal alpha defensin 2 expression (˜2.4×10⁵, ˜4×10⁵, and ˜1.6×10⁵, respectively) compared to −7.5×10⁴ in the saline control. Y134 and PHTPP were the most active agents for increasing the relative mRNA expression of beta defensin 1 in uterine cells (˜3×10⁶ and ˜1.05×10⁸, respectively compared to ˜1.1×10⁶ in the saline control), whereas ICI 182,780 and tamoxifen were most active for increasing the relative mRNA expression of beta defensin 1 in vaginal cells (˜2.15×10⁸ and ˜1.75×10⁸, respectively compared to ˜1.1×10⁸ in the saline control). Relative mRNA expression of beta defensin 2 in uterine cells was most significantly increased by PHTPP (˜1.55×10⁸ compared to ˜1×10⁵ in the saline control), whereas expression of the same in vaginal cells was not significantly affected by any of the SERMS or ER antagonists tested (i.e., ICI 182,780, tamoxifen, Y134, and PHTPP). Moreover, relative beta defensin 4 mRNA expression in uterine cells was most significantly increased by PHTPP and tamoxifen (˜8×10⁵ and ˜6×10⁵ compared to ˜0.4×10⁵ in the saline control), whereas tamoxifen significantly increased relative beta defensin 4 mRNA expression in vaginal cells (˜2.1×10⁸ compared to ˜1×10⁷ in the saline control).

Indeed, in addition to SLPI and defensins, it is expected that upon exposure to a SERM, epithelial cells will exhibit enhanced antimicrobial secretion, lowered proinflammatory cytokines/chemokines and co-receptor expression of CXCR4, CCR5 and CD4, enhanced intracellular protection by APOBEC3G/F, PKR, OAS, Mx1, and maintenance of barrier integrity. Exposure of NK cells to a SERM is also expected to enhance IFN-gamma production. Similarly, exposure of neutrophils to SERMs is expected to enhance antimicrobial secretion (e.g., lactoferrin and alpha-defensin), enhance ADCC, and lower proinflammatory cytokine/chemokine levels. Moreover, SERMs are expected to lower co-receptor and DC-SIGN expression and enhance intracellular protection in MACs/DCs.

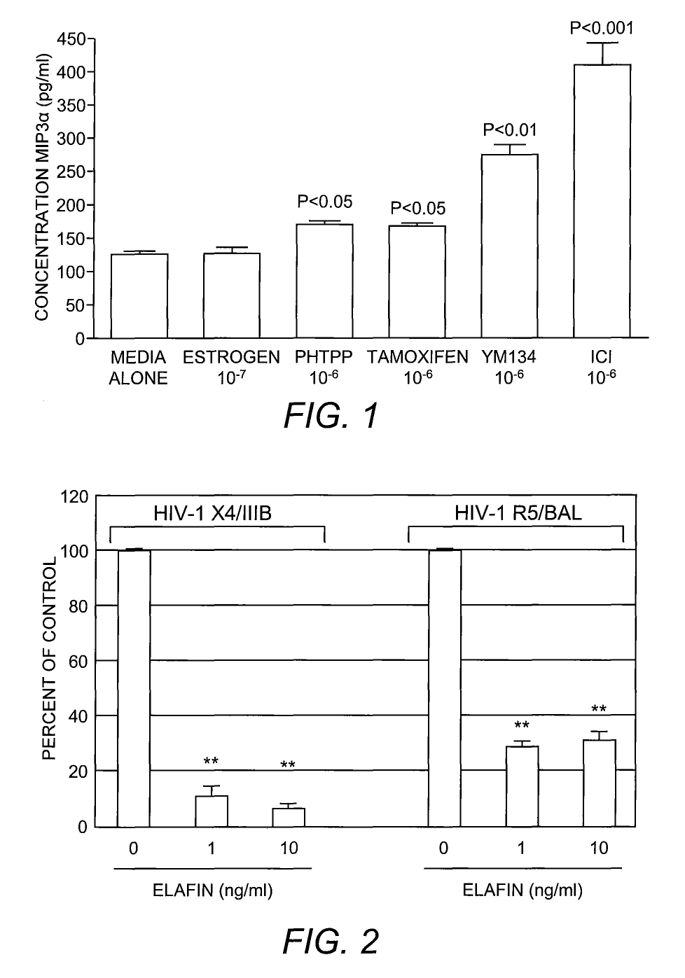

Indeed, FIG. 1 summarizes the effects of estradiol (estrogen) and four known SERMS on the secretion of MIP3 alpha (aka CCL20), a known potent endogenous antimicrobial peptide, by primary uterine epithelial cells in culture. Epithelial cells were isolated from the uteri of intact mice by enzymatic digestion prior to incubation at 37° C. in the presence or absence of known SERMS for 20 hours. Following incubation in 96-well plates, media was collected and assayed by ELISA for MIP3 alpha. Whereas estradiol had no effect on MIP3 alpha secretion at 20 hours and tamoxifen and PHTPP had a marginal effect, Y134 and ICI 182,780 added individually to the culture media significantly increased MIP3 alpha production. Of note, uterine stomal cells also inhibit secretion of MIP3 alpha from uterine epithelia.

Similar to the results with MIP3 alpha, the secretion of mouse KC (IL8, a potent neutrophil attractant and activator protein) from primary uterine epithelial cells was significantly increased upon 24-hour treatment with Y134 and ICI 182,270 added individually to the culture media, whereas PHTPP and tamoxifen had a marginal effect.

In addition to the analysis of MIP3 alpha and mouse KC, it was demonstrated that E₂ can attenuate LPS-induced TNFalpha secretion by female macrophages (Table 3), an effect which was blocked by the ERα/β antagonist ICI 182,780 (Table 4).

TABLE 3

| E₂ Concentration | TNFalpha |
|---|---|
| (nM) | (% of Control*) |
| 0.001 | 98.35 ± 3.19 |
| 0.01 | 97.72 ± 6.89 |
| 0.1 | 94.68 ± 11.62 |
| 1 | 82.00 ± 5.93^{†} |
| 10 | 82.14 ± 1.83^{†} |
| 100 | 81.06 ± 7.16^{†} |

*Control, no E₂.
N = 4.
^{†}p < 0.05 vs. Control.

TABLE 4

| | TNFalpha |
|---|---|
| Treatment | (% of Control*) |
| Control | 100 |
| E₂ | 73.96 ± 3.00 |
| ICI | 111.87 ± 15.68 |
| E₂ + ICI | 117.78 ± 16.63 |

*Control, vehicle.
N = 3.
p < 0.001 vs. vehicle.

These results were comparable to the effects seen with ERα- and ERβ-specific SERMS, Y134 and PHTPP, respectively (Table 5).

TABLE 5

| | TNFalpha |
|---|---|
| Treatment | (% of Control*) |
| Vehicle | 100 |
| E₂ | 73.96 ± 3.00 |
| Y134 | 100.25 ± 13.54 |
| E₂ + Y134 | 97.64 ± 18.78 |
| PHTPP | 79.96 ± 6.43 |
| E₂ + PHTPP | 117.84 ± 21.10 |

*Control, vehicle.
N = 3.
p < 0.05 vs. vehicle.

These results indicate that estradiol inhibits the production of MIP3α/CCL20, mouse KC and TNFalpha in vitro and in vivo. In contrast, ER antagonists (e.g., ICI 182,780 and Y134) stimulate MIP3α/CCL20, mouse KC and TNFalpha production. Therefore, the regulation of cytokines and chemokines produced by uterine epithelial cells occurs through ER-ligand interactions, particularly ERα, wherein selected SERMS can enhance antimicrobial protection against pathogens without inducing the recruitment of pro-inflammatory immune cells that might compromise reproductive function and/or increase the risk inflammation of the mucosae.

### Example 5

### Glucocorticoids Enhance the Human Systemic Inflammatory Response to Bacterial Endotoxin

Materials and Methods. Participants (n=36) were healthy male and female volunteers who were taking no chronic medications (other than oral contraceptives) and who were between the ages of 18 and 55.

On Day 1 of the study, participants were randomized in a double-blind manner to a Control Group, a Stress Group or a Pharm Group. A peripheral intravenous catheter was inserted in a hospital outpatient facility where participants remained during the day. For six hours, Control Group participants received intravenous saline at ml/hour by infusion pump; Stress Group participants received 1.5 μg/kg/minute hydrocortisone (SOLUCORTEF®, Upjohn) in normal saline at 10 ml/hour, and Pharm Group participants received 3.0 μg/kg/minute hydrocortisone in normal saline at 10 ml/hour. The following day (Day 2), participants were admitted to an acute care facility where a peripheral intravenous catheter was again inserted in a proximal arm vein. Continuous electrocardiographic and pulse oximetry measurements were initiated; blood pressure and core temperature were measured every 15 minutes for the next five hours and every 30 minutes for the next 4 hours. Participants received 10 ml/kg lactated Ringer's solution intravenously over the first 2 hours after which the infusion was decreased to 1.5 ml/kg/hour. After the first hour, participants were administered 2 ng/kg E. coli endotoxin intravenously over 2 minutes. Participants remained in the acute care facility for remainder of the day with continuous nursing care and under the direct supervision of a physician. The intravenous catheter was removed after 9 hours. The following day (Day 3) and 2 days later (Day 5), repeat blood samples were obtained by venipuncture.

Salivary free cortisol measurements were made on Day 1 to compare the in vivo cortisol pre-treatment with laboratory reports that typically test the effects of free glucocorticoid concentrations. On Day 2 of the study, total plasma cortisol was measured to allow for comparison with the many clinical reports that have assessed these hormones during experimental endotoxemia and in endotoxemic patients. For salivary free cortisol measurements, saliva samples were collected with a SALIVETTE™ collection device and stored at −80° C. until analysis by SALIMETRICS® Expanded Range High Sensitivity Salivary Cortisol Enzyme Immunoassay Kit (State College, Pa.). The analytical sensitivity of this assay is 3 ng/dL. For total cortisol, plasma was separated from heparinized whole blood and stored at −80° C. until analysis for plasma cortisol on an IMMULITE® 1000 analyzer using a Siemens Cortisol kit (Los Angeles, Calif.). The analytical sensitivity of this assay is 0.2 μg/dL. All adrenocorticotropic hormone (ACTH) measurements were performed on the IMMULITE® 1000 analyzer using a Siemens ACTH kit (Los Angeles, Calif.). The analytical sensitivity of this assay is 9 pg/mL. C-reactive protein (CRP) measurements were performed on the Roche P Module using the Roche High Sensitivity CRP kit (Indianapolis, Ind.). Analytical sensitivity of this assay is 0.1 mg/L. Complete blood counts were performed on the ADVIA 120® Hematology Analyzer using Siemens reagents (Ramsey, Minn.).

Plasma samples were collected and frozen at −80° C. for batched measurement of TNFalpha, IL-6 and IL-10 concentrations. TNFalpha was measured using a TNFalpha sandwich ELISA (paired antibodies; BD Biosciences Pharmingen, San Diego, Calif.). IL-6 levels were determined using an IL-6 ELISA kit (Peprotech, Rocky Hill, N.J.). IL-10 was measured using an IL-10 ELISA kit (Biosource, Camarillo, Calif.). Further cytokine testing was completed on frozen samples using multiplex immunobead technology (Luminex Corp.) which combines the principle of a sandwich immunoassay with fluorescent bead-based technology, allowing individual and multiplex analysis of multiple analytes in a single microtiter well. The multiplex plasma assay for 27 cytokines (IL-1beta, IL-1Ra, IL-2, IL-4, IL-5, IL-6, IL-7, IL-8, IL-9, IL-10, IL-12, IL-13, IL-15, IL-17, Eotaxin, FGF, G-CSF, GM-CSF, IFN-γ, IP-10, MCAF, macrophage inflammatory protein (MIP)-1alpha, MIP-1beta, PDGF, RANTES, TNF-alpha, and VEGF) was done in 96-well microplate format according to the protocol provided by the manufacturer (BioSource International, Camarillo, Calif.). Interassay variabilities for individual cytokines were in the range of 1.0% to 9.8% and intra-assay variabilities were in the range of 3.6% to 12.6%.

Cell staining and flow cytometry was also performed. Peripheral blood mononuclear cells from heparinized peripheral blood were isolated by FICOLL-HYPAQUE 1.077 (Sigma) gradient centrifugation. Cells were cultured with or without LPS (1 ng/ml; E. coli 0:113) at 37° C. for 1 hour and then washed twice in PBS/1% BSA/0.1% azide. Cells were stained for surface expression of CD163-PE (Mac2-158; Trillium Diagnostics, Bangor, MEP and CD14-FITC (TuK4; Caltag Laboratories, Burlingame, Calif.). Non-specific antibody binding was blocked by the addition of human IgG block (6 mg/ml; Sigma). Cells were washed and fixed in 1% formalin and analyzed by flow cytometry (FACSCAN, Beckton Dickinson). Monocytes were gated on forward/side scatter. Mean fluorescent intensity (MFI) was determined by the geometric mean of the fluorescence of gated monocytes.

The primary end-point of this study was the human in vivo cytokine response to LPS as assessed by ELISA determination of 3 different plasma cytokines: the pro-inflammatory cytokine TNF-alpha, which is transiently released after LPS; the more pleiotropic cytokine IL-6, which has largely pro-inflammatory effects and which persists in plasma for several hours after LPS; and the anti-inflammatory cytokine IL-10, which peaks at 3 hours after LPS administration. It was expected that pre-treatment of participants with a 6-hour infusion of cortisol would induce an increased plasma cytokine (TNF-alpha, IL-6) response to the intravenous LPS. Power analysis was based on finding differences in plasma TNF-alpha and IL-6 concentrations at 2 hours post-LPS exposure between participants in the Control Group and participants in the Stress Group based on previously published data (Barber, et al. (1993) J. Immunol. 150:1999-2006). Using these data, a Type I error of 0.025 was calculated to account for two comparisons and power of 0.90 with 12 subjects per group. Non-parametric rank tests and Student's t-tests, with appropriate transformation to normality, were used to compare control and treatment groups at each time period. More advanced methods were used to account for the correlated nature of the data that resulted from multiple measures on each subject over time. Generalized estimating equations (GEE) were used as the primary analytic tool to consider all time points and to characterize changes over time.

Results. All study participants completed the protocol as planned. Participants were evenly matched between the 3 groups with regard to sex, age and weight (Table 6).

TABLE 6

| | Sex | Age | Weight |
|---|---|---|---|
| Group | (M/F) | (Years) | (kg) |
| Control | 5/6 | 35.5 (4.3) | 75.8 (3.6) |
| Stress | 5/7 | 33.0 (2.5) | 78.7 (4.1) |
| Pharm | 6/7 | 36.3 (3.5) | 77.5 (3.7) |

Control = intravenous saline;
Stress = intravenous hydrocortisone @ 1.5 μg/kg/min for 6 hours;
Pharm = pharmacologic hydrocortisone @ 3 μg/kg/min for 6 hours.

LPS induced the expected clinical response which included an increase in heart rate of approximately 30 beats/minute and increase in body temperature to approximately 38.5° C. (Table 7).

TABLE 7

| | CONTROL | STRESS | PHARM |
|---|---|---|---|
| BASE HR | 72 | 73 | 70 |
| (bpm) | (1.8) | (2.4) | (3) |
| PEAK HR | 101 | 111 | 106 |
| (bpm) | (2.9) | (4.4) | (4) |
| BASE T | 36.5 | 36.6 | 36.5 |
| (° C.) | (0.17) | (0.12) | (0.1) |
| PEAK T | 38.4 | 38.4 | 38.1 |
| (° C.) | (0.11) | (0.14) | (0.11) |
| BASE WBC × | 6.3 | 6.8 | 7.0 |
| 10³ per ml | (0.4) | (0.5) | (0.3) |
| 4 Hour WBC × | 10.3 | 9.1 | 9.1 |
| 10³ per ml | (0.9) | (0.6) | (1.0) |
| BASE MO | 300 | 297 | 264 |
| per ml | (23) | (33) | (34) |
| 4 Hour MO | 158 | 112 | 85 |
| per ml | (30) | (19) | (16) |
| BASE CD14⁺ | 18.6 | 17.1 | 17.2 |
| % PBMNCs | (1.1) | (1.4) | (1.4) |
| 4 Hour CD14⁺ | 36.6 | 17.9* | 17.6* |
| % PBMNCs | (5) | (3.4) | (2.7) |
| Base PMN | 3770 | 4312 | 4842 |
| per ml | (301) | (449) | (385) |
| 4 Hour PMN | 9705 | 8353 | 8390 |
| per ml | (894) | (575) | (980) |
| BASE CRP | 1.9 | 2.6 | 1.8 |
| (mg/L) | (0.7) | (0.9) | (1.1) |
| 24 Hour CRP | 40.4 | 51.0 | 44.0 |
| (mg/L) | (4.7) | (4.6) | (3.4) |

There were no significant differences between groups in any of these measurements. Fever was preceded in all cases by headache, myalgia and chills. Several subjects reported transient nausea. Hypotension (systolic arterial blood pressure<100 mm Hg) was not observed in any study participant.

Salivary free cortisol concentrations on the morning of Day 1 were similar in all 3 groups. Salivary free cortisol concentrations decreased during the day in the Control Group and rose in the Stress and Pharm Groups to approximately the expected concentrations at the end of the 6-hour intravenous infusion of hydrocortisone. The following morning (Day 2) before administration of LPS, plasma cortisol concentrations were similar in all 3 groups. The plasma cortisol response after LPS administration was again similar in all 3 groups with the expected peak at 4 hours (Rassias, et al. (2005) Crit. Care Med. 33(3):512-9)). The plasma ACTH response also peaked at 3-4 hours after LPS (Schreiber, et al. (1993) Neuroendocrinology 58:123-128). ACTH concentrations were higher in the Stress Group compared to the Control Group 3 and 4 hours after LPS injection.

Plasma concentrations of cytokines were measured immediately before LPS administration and at 2, 3, 4 and 8 hours after LPS. Values of all cytokines were also measured 24 hours after LPS in the first 21 subjects and were found to be negligible or below detection limits. TNF-alpha concentrations in plasma were highest at 2 hours, declined rapidly thereafter, and were not significantly different between groups. Plasma IL-6 also reached a peak concentration 2 hours after LPS, remained elevated at 3 and hours after LPS administration, and was significantly higher in the Stress Group compared to the Control Group (p<0.005), wherein there was no difference between the Control Group and the Pharm Group. Plasma IL-10 peaked in all groups at 3 hours after LPS and was significantly lower in the Stress Group compared to the Control Group (p<0.05), wherein there was no difference between the Control Group and the Pharm group. Subsequently, unthawed plasma samples were tested for concentrations of circulating inflammatory response mediators using multiple immunobead methodology. These tests confirmed the ELISA results for TNF-alpha, IL-6 and IL-10 and also showed a significant augmentation of plasma G-CSF concentrations in the Stress Group compared to Control Group values with no difference between the Pharm and Control Groups. Intergroup comparisons of the plasma concentrations of all other mediators were not different between groups.

Peripheral blood leukocyte counts were measured immediately before administration of LPS (Day 2) and 4 hours after LPS administration (Day 2). There were no significant differences between groups in either total peripheral blood white cell counts or in the percentage of cells that were neutrophils (Table 7). C-reactive protein increased substantially on the morning of Day 3 of the study with no differences between groups (Table 7).

### Example 6

### Genetically Engineered Lactobacillus

In women, commensal bacteria such as Lactobacilli play a key role in reproductive health by contributing to an environment that minimizes the ability of pathogens to infect both the lower and upper FRT. Protection is derived from the secretion of lactic acid, the production of bacteriocins, and the stimulation of endogenous antimicrobials that inhibit pathogen replication. Lactobacilli have been engineered to secrete therapeutic proteins, antibodies, antigens and coreceptors with the capability of interfering with viral infectivity. In this regard, it has shown that it is possible to inhibit HIV infectivity by expressing specific CD4 protein receptors for HIV in a Lactobacillus species commonly found in the vaginal microflora (Theresa, et al. (2003) Proc. Natl. Acad. Sci. USA 100:11672). Therefore, to block the heterosexual transmission of HIV, the present invention embraces the use of hormone-responsive engineered human vaginal isolates of Lactobacillus to provide mucosal immune protection against HIV and other STI in the FRT throughout the menstrual cycle and following menopause.

In carrying out this embodiment of the invention, Lactobacilli normally found in the human vagina are engineered to respond to human hormones and secrete anti-microbial agents throughout the menstrual cycle without compromising normal reproductive function or increasing the risk of HIV infection. Lactobacilli are engineered with estrogen (ER) or progesterone (PR) receptors, and estrogen response elements (ERE) or progesterone response elements (PRE) located upstream of coding sequences for anti-HIV agents. Upon an increase in estrogen or progesterone level, the recombinant Lactobacillus selectively responds by secreting molecules that are antiviral against HIV.

Unique characteristics of this approach include the use of the endocrine system to optimize FRT endogenous immune protection that protects against viral pathogens including HIV. This approach differs from conventional approaches by focusing on endogenously produced microbicides known to exhibit anti-microbial activity. Furthermore, in so far as it has been appreciated that sex hormones regulate immune protection in the upper and lower FRT, hormonal regulation of anti-HIV agent expression will ensure that both of these potential sites of HIV infection are targeted.

Antivirals are expressed by lactobacilli by introducing coding sequences for the anti-viral agent into a shuttle vector such as pTSV2. For example, the coding sequences for antivirals can be introduced into the unique restriction sites EcoRI and BamHI of the pTSV2 plasmid so that the signal peptide Usp45 from L. lactis is expressed in-frame to achieve high secretion efficiency of the recombinant protein into the bacterial supernatant. FI secretion plasmids and control plasmid pTSV2 are transformed into Lactobacilli by electroporation. Antiviral activity of Lactobacillus-derived microbicides is tested against laboratory strains of HIV (IIIB and Bal) as well as primary HIV-1 isolates using the TZM infection assay. This approach is used to evaluate individual antimicrobials (SLPI, Elafin, HBD2, MIP3alpha, etc.) for their ability to be secreted by the engineered Lactobacilli.

To develop bacteria that are hormonally responsive, the DNA sequences of the human ER and PR genes are integrated into the Lactobacillus genome. When a strain of Lactobacillus contains both a hormonal response element and the corresponding hormone receptor, expression of the antimicrobial agent is achieved. To ensure hormone-responsiveness, additional constructs can be engineered that contain EREs and PREs which control expression of the reporter gene Lac-Z. In the presence of hormone, beta-galactosidase is synthesized and secreted into the medium. This system provides a means to readily determine hormone-responsiveness of the promoter elements.

### Example 7

### Trappin-2/Elafin Activity and Genetically Engineered E. coli Expressing the Same

Trappin-2/Elafin is a serine protease inhibitor that plays a major role as an anti-inflammatory mediator at mucosal surfaces. In addition, Trappin-2/Elafin has antibacterial activity against gram-positive and gram-negative bacterial and fungal pathogens. Using a general gram-positive vector-APU, which utilizes the SecA secretory system, export of Elafin protein (GENBANK Accession No. NP_{—}002629) through the Twin arginine translocase (Tat) system was analyzed. The lactobacillus USP45 signal peptide of the APU vector was replaced by the signal peptide from the YcdB protein found in E. coli. Since the Tat system exports only proteins folded in their mature conformation, this system eliminates potential pitfalls associated with the dependence of export as a random coil as required for the SecA system.

E. coli cultures containing either no vector or YcdB-Elafin sequences were grown overnight and the cultures centrifuged for 10 minutes at 3400×g (2° C.) and the supernatants (0.5 ml) passed over an ultrafree centrifugal filter (30,000 molecular weight cutoff) to remove a majority of endotoxin. Samples were then assayed for Elafin by ELISA. The results of this analysis indicated that more than 60 pg/ml Elafin was secreted by engineered E. coli, indicating that a commensal bacterium can secrete a human reproductive tract endogenous microbicide.

To demonstrate efficacy of Elafin, the production of Trappin-2/Elafin by epithelial cells from the human upper and lower female reproductive tract was analyzed as was the anti-HIV-1 activity of Trappin-2/Elafin.

Materials & Methods. A total of 11 different patients were used to obtain epithelial cells from the uterus, Fallopian tube, endocervix and ectocervix. Epithelial cells were isolated according to known methods (Fahey, et al. (1999) Am. J. Reprod. Immunol. 42(1):49-57; Fahey et al. (1998) Immunol. Invest. 27(3):167-80). Briefly, tissues were rinsed with 1×PBS and minced into 1-2 mm fragments prior to subjecting them to enzymatic digestion for 2 hours at 37° C. The enzyme mixture contained 3.4 mg/ml pancreatin (Invitrogen Life Technologies, Carlsbad, Calif.), 0.1 mg/ml hyaluronidase (Worthington Biochemical, Lakewood, N.J.), 1.6 mg/ml collagenase (Worthington Biochemical), and 2 mg/ml D-glucose, in 1×HBSS (Invitrogen Life Technologies). After digestion, cells were dispersed through a 250-mm mesh screen, washed, and resuspended in DMEM/F12 complete medium without phenol red, supplemented with 20 mM HEPES, 2 mM L-glutamine (all from Invitrogen Life Technologies), 50 μg/ml primocin (Invivogen, San Diego, Calif.), and 10% defined FBS (Hyclone, Logan, Utah). Epithelial sheets were separated from stromal cells by filtration through a 20-mm nylon mesh filter (Small Parts, Miami Lakes, Fla.). Epithelial cell sheets retained on the filter were recovered by rinsing and backwashing, centrifuged at 500×g for 10 minutes and analyzed for cell number and viability. By this procedure, epithelial cells that stain positive for the epithelial antigens Ber-EP4 and cytokeratin and negative for CD4, CD45, and vimentin were isolated (Wallace, et al. (2001) J. Steroid Biochem. Mol. Biol. 76(1-5):203-11).

To establish a cell culture system of polarized human uterine (UT), Fallopian tube (FT), and endocervical epithelial cells (Cx) with both apical and basolateral compartments, primary cells were cultured in Human Extracellular Matrix (Becton Dickenson, Franklin Lakes, N.J.)-coated Falcon cell culture inserts in 24-well culture plates (Fisher Scientific, Pittsburgh, Pa.). For these experiments, apical and basolateral compartments contained 300 and 850 μl of complete medium, respectively. In order to keep the culture conditions similar, the same procedure was followed for culturing the squamous ectocervical epithelial cells (Ecx), which do not polarize. The medium was changed every 2 days. The cells were treated with 25 μg/ml TLR3 agonist Poly(I:C) (Invivogen) for 24 hours after which apical and basolateral conditioned media was collected and centrifuged for 5 minutes at 10,000×g and stored at −80° C. until use.

Tight junction formation of cultured epithelial cell monolayers was assessed by periodically measuring transepithelial resistance (TER) using an EVOM electrode and Voltohmmeter (World Precision Instruments, Sarasota, Fla.) (Richardson, et al. (1995) Biol. Reprod. 53(3):488-98). TER is a functional measurement of the integrity of tight junctions in an epithelial cell monolayer. Since the presence of non-epithelial cells in the culture interferes with the formation of tight junctions and therefore prevents an increase in TER. TER is also an indicator for the purity of the epithelial monolayer.

Concentrations of Trappin-2/Elafin in the apical and basolateral supernatants from primary FRT epithelial cells and CVL from HIV(+) and (−) women were determined with an ELISA Duoset kit (R&D Systems, Minneapolis, Minn., USA) according to the manufacturer's protocol. This assay measures both Trappin-2 and Elafin. Amounts of Trappin-2/Elafin were measured based on a standard curve after OD measurements at 450 nm on an ELISA reader (Dynex, Chantilly, Va., USA).

Real-time RT-PCR was carried out with a two-step protocol (Godfrey, et al. (2000) J. Mol. Diagn. 2(2):84-91). Total RNA was isolated from cells using TRIZOL Reagent according to the manufacturer's recommendations (Invitrogen Life Technologies) and purified with RNEASY columns (Qiagen, Valencia, Calif., USA). Coincident with RNA purification was on-column DNase digestion using the RNase-Free DNase set (Qiagen). For each specimen, 400 ng of total RNA was reverse-transcribed using the ISCRIPT cDNA synthesis kit (Bio-Rad, Hercules, Calif., USA) according to the manufacturer's recommendations in a 20 μl volume. Relative mRNA expression levels of Trappin-2/Elafin were measured using the 5′ fluorogenic nuclease assay in real-time quantitative PCR using TAQMAN chemistry on the ABI 7300 Prism real-time PCR instrument (Applied Biosystems, Foster City, Calif., USA). The Trappin-2/Elafin and β-actin primer/MGB probe sets were obtained from Applied Biosystems assays-on-demand (ID nos. Hs00160066_ml and 4333762T, respectively). This primer-probe set recognizes both Trappin-2 and Elafin. PCR was conducted using the following cycle parameters: 95° C., 12 minutes for 1 cycle (95° C., 20 seconds; 60° C., 1 minute), for 40 cycles. Analysis was conducted using the sequence detection software supplied with the ABI 7300. The software calculates the threshold cycle (Ct) for each reaction which was used to quantify the amount of starting template in the reaction. The Ct values for each set of duplicate reactions were averaged for all subsequent calculations. A difference in Ct values (ΔCt) was calculated for each gene by taking the mean Ct of each gene of interest and subtracting the mean Ct for the housekeeping gene β-actin for each cDNA sample. Assuming that each reaction functions at 100% PCR efficiency, a difference of one Ct represents a 2-fold difference. Relative expression levels were expressed as a fold-increase in mRNA expression and calculated using the formula 2^{-ΔΔCt}.

The TZM indicator cell line is a HeLa cell derivative that expresses high levels of CD4, CCR5 and CXCR4 (Wei, et al. (2002) Antimicrob. Agents Chemother. 46(6):1896-905). The cells contain HIV long terminal repeat (LTR)-driven β-galactosidase and luciferase reporter cassettes that are activated by HIV tat expression. TZM cells were routinely sub-cultured every 3 to 4 days by trypsinization and were maintained in TZM media consisting of DMEM (Invitrogen Life Technologies) supplemented with 10% defined FBS (HyClone, Logan, Utah, USA), 2 mM L-glutamine (Invitrogen Life Technologies), and 50 mg/ml primocin (Invivogen, San Diego, Calif., USA) and did not contain phenol red.

TZM cells were seeded at 2×10⁴ cells per well in a 96-well microtiter plate and allowed to adhere overnight at 37° C. Varying doses of recombinant human Trappin-2/Elafin (Peprotech, Rocky Hill, N.J.) were incubated with HIV-1 IIIB and BaL at multiplicity of infection (MOI) 1 for 1 hour at 37° C. in a final volume of 100 μl. Following incubation, the media was aspirated from TZM cells and the virus plus Trappin-2/Elafin was added to the cells along with 100 μl of TZM media. Luciferase activity was measured after 48 hours at 37° C. with 5% CO₂ in a humidified incubator. Briefly, the supernatants were aspirated, and the cells were lysed with a BETA GLO luciferase assay substrate. Light intensity of each well was measured using a luminometer. Uninfected cells were used to determine background luminescence. All infectivity assays were performed in quadruplicate.

Other experiments were conducted in order to determine whether the inhibitory effects of Trappin-2/Elafin were at the cell surface level such as the blocking of a co-receptor. For these experiments, varying doses of Trappin-2/Elafin were directly added to TZM cells and incubated for 1 hour at 37° C. prior to washing with 1×PBS and addition of IIIB or BaL viruses. In additional experiments to determine possible post-infection mechanisms of inhibition by Trappin-2/Elafin, TZM cells were infected with HIV-1 IIIB and BaL at MOI 1 and were washed at 6 hours and 24 hours post-infection followed by the addition of 10 ng/ml rTrappin-2/Elafin. Assays were developed at 48 hours by addition of the BETA-GLO substrate and measuring relative light units using a luminometer.

Viability of TZM cells upon treatment with Trappin-2/Elafin and CVL was quantified using the CELLTITER 96° AQᵤₑₒᵤₛ One Solution Cell Proliferation Assay (Promega) according to manufacturer's instructions. Briefly, reagent was added directly to cell cultures and incubated for 1 hour at 37° C. followed by reading the plate in a plate reader at OD 490 nm.

CVL samples from 32 HIV(+) women (12 Black, 9 Hispanic, and 12 White) and 15 HIV(−) women (5 Black, 5 Hispanic, and 5 White) were obtained. CVL from women were cataloged by race based on self-identification. The HIV(+) and (−) women were in the same age range (18-50 yrs). The HIV(+) women were relatively healthy with average CD4 counts of 712 cells/mm³ blood, average plasma viral load of 12,666 copies/ml and not on any anti-retroviral therapy. Only 6 out of 32 showed detectable genital tract viral load. CVL was collected by washing the cervical-vaginal area with 10 cc of sterile saline (pH 7.0) and collecting the fluid which was then centrifuged at 10,000×g for 5 minutes and separated from the cellular fraction. The supernatants were aliquoted and stored in −80° C. freezer until use. For the HIV(−) samples used for this study, CVL were collected and frozen immediately at −80° C. Prior to assaying the supernatants, samples were thawed, centrifuged at 10,000×g for 5 minutes and separated from the cellular fraction.

Results. This analysis indicated that primary uterine, Fallopian tube, cervical and ectocervical epithelial cells produce Trappin-2/Elafin constitutively. Specifically, Fallopian tube had the highest levels of Trappin-2/Elafin expression, 10- to 368-fold higher than that seen in Ecx, set at 1. Trappin-2/Elafin mRNA levels in the cervix were also greater than the ectocervical epithelial cells, 2- to 36-fold higher. Uterine epithelial cells, however, typically showed very low Trappin-2/Elafin mRNA expression, significantly lower than epithelial cells from all the other compartments (Fallopian tube, cervical and ectocervical epithelial cells)

It has been shown that the FRT epithelial cells can mount an antiviral response upon stimulation with Poly(I:C), a synthetic mimic for viral dsRNA. Therefore, it was of interest to determine whether Trappin-2/Elafin, a known anti-microbial, would also be produced in response to Poly(I:C) stimulation. This analysis indicated that when uterine epithelial cells were treated with Poly(I:C) for 24 hours, Trappin-2/Elafin mRNA expression was significantly upregulated 4-fold to 95-fold when compared to control cells whose expression was set at 1 (6 out of 6 patients). In a time-course experiment where cells were treated with Poly(I:C) and harvested at 3, 6, and 24 hours post-treatment, it was observed that Poly(I:C) treatment upregulated Trappin-2/Elafin mRNA expression at 6 hours with continued increases seen at 24 hours. To demonstrate whether Poly(I:C) also stimulated Trappin-2/Elafin protein secretion, 24 hour conditioned media was tested by ELISA. It was found that Trappin-2/Elafin secretion by UT epithelial cells was significantly increased upon Poly(I:C) stimulation. Further, when apical and basolateral secretions were analyzed, it was observed that the secretion of Trappin-2/Elafin was preferentially apical. Trappin-2/Elafin in basolateral secretions was measurable but very low relative to apical secretions.

To more fully evaluate the extent of Poly(I:C) mediated Trappin-2/Elafin secretion throughout the FRT, similar analyses were carried out with Fallopian tube, endocervix, and ectocervix epithelial cells. Unexpectedly, it was found that whereas cells from all compartments constitutively produced Trappin-2/Elafin both at the mRNA and the protein level, Poly(I:C) stimulation enhanced RNA expression and protein secretion in only a fraction of the patients tested. Out of 4-6 patients tested for each compartment, approximately ⅓ typically responded to Poly(I:C) by upregulating Trappin-2/Elafin.

Trappin-2/Elafin is a known anti-bacterial molecule that has been shown to be effective against both gram-positive and gram-negative bacteria. In so far as it was demonstrated that a synthetic double-stranded viral RNA (dsRNA) analog Poly(I:C) enhanced Trappin-2/Elafin production/secretion from FRT epithelial cells, it was determined whether Trappin-2/Elafin could have direct antiviral activity. Since HIV-1 is an important sexually transmitted pathogen, the activity of recombinant Trappin-2/Elaf in (rTrappin-2/Elafin) was evaluated against HIV-1 X4/T-tropic IIIB and R5/M-tropic BaL. HIV-1 IIIB and BaL were incubated with rTrappin-2/Elafin at 0.01, 0.1, 1, and 10 ng/ml for 1 hour at 37° C. TZM-bl indicator cells were plated the previous day at 25,000 cells per well and grown to 70-80% confluence. The virus-Trappin-2/Elafin mixture was added to the TZM cells and incubated for 48 hours at 37° C. At the end of the incubation period, BETA-GLO substrate was added to the cells and viral infection was quantified using a luminometer, expressed as relative light units (RLU). The data was expressed as percent of control with virus-only control set at 100%. This analysis indicated that rTrappin-2/Elafin significantly inhibited both IIIB and BaL at all the concentrations tested, in the range of 80% inhibition of IIIB and 60% inhibition of BaL (FIG. 2). It was demonstrated by ELISA that the biological concentrations of Trappin-2/Elafin secreted by epithelial cells, both constitutively and upon Poly(I:C) stimulation, ranged between 0.25-9 ng/ml. Therefore, the concentrations of Trappin-2/Elafin showing anti-HIV-1 activity were in the range of physiological levels of this molecule that were secreted by the FRT epithelial cells. Since the inhibitory activity was observed as a result of pre-incubation of HIV-1 and rTrappin-2/Elafin, it is contemplated that the effect of Trappin-2/Elafin on viral infection was direct. Viability studies were conducted in parallel that ensured that the inhibitory activity observed was not due to toxic effect of rTrappin-2/Elafin on the TZM cells.

Anti-HIV factors have been shown to inhibit HIV by multiple mechanisms, including by direct interaction with HIV, by blocking cell surface receptors (CXCR4, CCR5), and by affecting post-infection steps. To demonstrate whether rTrappin-2/Elafin might also have indirect effects on HIV-1 infection by blocking any cell-surface receptors or molecules, the TZM cells were pre-incubated with 0.1 and 1 ng/ml of rTrappin-2/Elafin for 1 hour at 37° C. Following incubation cells were washed repeatedly with media prior to addition of HIV-1 IIIB and BaL after which the cells were incubated for 48 h and infectivity was assessed. In contrast to the pre-incubation experiments described above, it was observed that pre-exposure of the cells to Trappin-2/Elafin at any of the doses tested had no effect on infectivity of TZM cells by IIIB and BaL. This indicated that the inhibitory activity of Trappin-2/Elafin was through a direct interaction with the virus rather than at the level of the target cell surface, for example, through the blocking of receptors.

To determine whether Trappin-2/Elafin acts through post-infection mechanisms in addition to directly interacting with virus, TZM cells were infected with IIIB and/or BaL, washed at 6 and 24 hours post-infection to remove free virus, after which rTrappin-2/Elafin (1 ng/ml) was added to TZM cells. Other than a slight inhibition observed at 24 hours post-infection with the IIIB virus, no significant post-infection inhibition was observed. Overall these data indicate that the inhibitory activity of Trappin-2/Elafin was through direct interactions with the virus rather than at the level of the cell surface, or through the disabling of post-infection steps.

Because these experiments indicated that antiviral activity might be due to epithelial cell production of Trappin-2/Elafin, studies were undertaken to remove Trappin-2/Elafin by antibody neutralization. To ensure that the antibody used was sufficient to remove Trappin-2/Elafin, known amounts of Trappin-2/Elafin were first neutralized. This analysis indicated that neutralization of rTrappin-2/Elafin (1 ng/ml) was possible, with a complete reversal of anti-HIV activity. However, when an attempt was made to neutralize secretions from primary epithelial cell cultures, known to contain amounts of Trappin-2/Elafin (0.1 ng/ml), a statistically significant 20% reversal was obtained. This finding fits with several studies showing that secretions from the FRT contain between 12-20 known antimicrobial factors, many of which have anti-HIV-1 activity (Ghosh, et al. (2008) Fertil. Steril. 89(5 Suppl):1497-506; Schaefer, et al. (2005) J. Immunol. 174(2):992-1002; Fahey & Wira (2002) J. Infect. Dis. 185(11):1606-13; Keller et al. (2007) AIDS 21(4):467-76; Venkataraman et al. (2005) J. Immunol. 175(11):7560-7; Fahey, et al. (2005) Hum. Reprod. 20(6):1439-46). These results indicate that Trappin-2/Elafin produced by human uterine epithelial cells in culture is responsible for some of the antiviral activity measured in apical secretions.

To determine whether Trappin-2/Elafin might be important for protection in vivo, Trappin-2/Elafin levels were measured in cervico-vaginal lavages (CVL) from both HIV(+) and HIV(−) women. Trappin-2/Elafin protein was found in CVL from both groups, ranging from 4-8 ng/ml. Moreover, while not statistically different, Trappin-2/Elafin levels in HIV(−) women tended to be higher than that measured in HIV(+) women. Differences did not reach statistical significance, possibly due to variation within patient groups (p=0.09). The higher levels of Trappin-2/Elafin measured in HIV(−) women might indicate a protective role which is compromised when levels are lowered upon infection. When the data was stratified according to race, no significant differences were found when HIV(−) Black, Hispanic, and White women were compared to HIV(+) women in terms of Trappin-2/Elafin levels. Within each racial group, Trappin-2/Elafin levels were higher in the HIV(−) CVL when compared to the HIV(+) CVL. Again, the differences did not reach statistical significance, possibly due to the variability among patients despite a general trend toward elevated values in the HIV(−) women compared to the HIV(+) women. When the HIV(+) CVL was stratified according to menstrual status, a significant increase of Trappin-2/Elafin secretion was observed in the secretory phase of the cycle suggesting that this molecule might be hormonally regulated. The presence of Trappin-2/Elafin in CVL indicates that Trappin-2/Elafin may be a relevant molecule for in vivo protection against HIV-1.

### Example 8

### Anti-HIV Activity of CCL20/MIP3alpha

Materials and Methods. Source of Tissues Human uterine and Fallopian tube tissues were obtained from women undergoing hysterectomy. Tissues used in this study were collected from patients with benign conditions such as fibroids distal from the site of pathology. The sections were examined by a pathologist and proclaimed to be free of pathological lesions. A total of nine different patients were used to obtain uterine epithelial cells and a total of seven patients were used to obtain Fallopian tube epithelial cells.

Epithelial cells were isolated according to known methods (Fahey, et al. (1999) supra; Fahey, et al. (1998) supra) as described herein.

To establish a cell culture system of polarized human uterine epithelial cells (UTEC) and Fallopian tube epithelial cells (FTEC) with both apical and basolateral compartments, primary human UTEC and FTEC were cultured in Human Extracellular Matrix (Becton Dickenson, Franklin Lakes, N.J., USA)-coated Falcon cell culture inserts in 24-well culture plates (Fisher Scientific, Pittsburgh, Pa., USA). For these experiments, apical and basolateral compartments contained 300 and 850 μL of complete medium, respectively. The medium was changed every 2 days. Cells were treated with the following TLR agonists for 24 hours: ultrapure LPS from Escherichia coli (List Biological Laboratories, Campbell, Calif., USA), 1 pg/mL; Poly(I:C) (Invivogen), 25 μg/mL; zymosan from Saccharomyces cerevisiae (Invivogen), 100 ng/mL and Flagellin from E. coli (Inotek Pharmaceuticals), 100 ng/mL. After 24 hour incubation, apical and basolateral conditioned media were collected and centrifuged for 5 minutes at 10,000×g and stored at) 80° C. until use.

Tight junction formation of cultured epithelial cell monolayers was assessed by periodically measuring transepithelial resistance (TER) using an EVOM electrode and Voltohmmeter (World Precision Instruments, Sarasota, Fla., USA).

Concentrations of CCL20/MIP3α in the apical and basolateral supernatants from human primary UTEC and FTEC were determined with an ELISA Duoset kit (R&D Systems, Minneapolis, Minn., USA) according to the manufacturer's protocol. Amounts of CCL20/MIP3α were quantified based on a standard curve after OD measurements at 450 nm on an ELISA reader (Dynex, Chantilly, Va., USA).

Real-time RT-PCR was carried with a two-step protocol as described herein. The CCL20/MIP3a and β-actin primer/MGB probe sets were obtained from Applied Biosystems assays on-demand (ID nos. Hs00171125 and 4333762T, respectively). PCR was conducted using the following cycle parameters: 95° C., 12 minutes for 1 cycle (95° C., 20 seconds; 60° C., 1 minute), for 40 cycles. Analysis was conducted using the sequence detection software supplied with the ABI 7300. As indicated herein, the software calculates the threshold cycle (Ct) for each reaction which was used to quantify the amount of starting template in the reaction.

The TZM-bl assay was as described herein. Varying doses of recombinant human CCL20/MIP3α (Peprotech, Rocky Hill, N.J., USA) were incubated with HIV-1 IIIB and HIV-1 BaL at multiplicity of infection (MOI) 1 for 1 hour at 37° C. in a final volume of 100 μL. Following incubation, the media was aspirated from TZM cells and the virus plus CCL20/MIP3α was added to the cells along with 100 μL of TZM media. Luciferase activity was measured as described herein after 48 hour at 37° C. with 5% CO₂ in a humidified incubator. All infectivity assays were performed in quadruplicate.

Other experiments were conducted in order to determine whether the inhibitory effects of CCL20/MIP3α were at the cell surface level such as the blocking of a co-receptor. For these experiments, varying doses of CCL20/MIP3α were directly added to TZM cells and incubated for 1 hour at 37° C. prior to washing out with 1×PBS and addition of IIIB or BaL viruses. In additional experiments to determine possible post-infection mechanisms of inhibition by CCL20/MIP3α, TZM cells were infected with HIV-1 IIIB and BaL at MOI 1 and were washed out at 6 and 24 hour post-infection followed by the addition of 20 ng/mL CCL20/MIP3α. Assays were developed at 48 hours by addition of the BETA-GLO substrate and measuring relative light units using a luminometer.

Statistical analysis included a two-tailed paired t-test or a one-way anova with Bonferonni's post-test using Graph-Pad InStat version 3.0a (GraphPad Software, San Diego, Calif., USA). A P-value of <0.05 was taken as indicative of statistical significance.

Results. Epithelial cells were isolated from endometrium and Fallopian tube and grown to confluence and high TER on inserts in TRANSWELL plates. The cells were then treated with the following pathogen-associated molecular patterns: LPS (TLR 4 agonist), Poly(I:C) (TLR 3 agonist), Zymosan (TLR1/2 or 2/6 agonist) and Flagellin (TLR 5 agonist) for 24 hours. Following treatment, RNA was isolated from the cells and analyzed by TAQMAN real-time RT-PCR. Using one representative patient sample, it was observed that only TLR3 agonist Poly(I:C) treatment enhanced CCL20/MIP3α mRNA production by the cells by greater than two-fold. Since Poly(I:C) treatment was the only treatment that upregulated CCL20/MIP3α mRNA, multiple patient samples were analyzed to see if the upregulation was consistent among patients. In five out of five patients, enhancement of CCL20/MIP3α mRNA production was observed upon Poly(I:C) treatment. Although there was variability among patients, in all five, CCL20/MIP3α mRNA upregulation was greater than two-fold which was used as the cut-off for significance.

To observe whether epithelial cells isolated from Fallopian tubes produce CCL20/MIP3α, these cells were cultured and treated with Poly(I:C) for 24 hours. Although four out of four patient samples expressed CCL20/MIP3α mRNA, upregulation upon Poly(I:C) treatment was observed in only two out of four samples.

Observing that CCL20/MIP3α mRNA expression was induced upon Poly(I:C) treatment, CCL20/MIP3α protein secretion was quantified from both endometrium and Fallopian tube epithelial cells. Epithelial cells were isolated and cultured on inserts in TRANSWELL plates until confluent with high TER, as described above, and treated with Poly(I:C). After 24 hour treatment, both the apical and the basolateral conditioned media were harvested, centrifuged to remove debris and assayed for CCL20/MIP3α protein by ELISA. The results of this analysis indicated that the endometrium and Fallopian tube epithelial cells constitutively secrete CCL20/MIP3α protein into the apical compartment with Fallopian tube samples generally secreting a higher quantity. The levels ranged from 0.24 to 12.48 ng/mL. When cells were treated with Poly(I:C) for 24 hours, there was significant increase in both the endometrium and Fallopian tube CCL20/MIP3α secretion. In other studies comparing apical with basolateral secretion, it was found that most if not all CCL20/MIP3α was secreted apically, in the direction of the FRT lumen.

The data herein showed that CCL20/MIP3α could be induced by the viral stimuli Poly(I:C). As CCL20/MIP3α is a known anti-bacterial molecule that has been shown to be effective against both gram-positive and gram-negative bacteria, it was determined whether CCL20/MIP3α could be virucidal as well. The activity of recombinant CCL20/MIP3α (rCCL20/MIP3α) was tested against HIV-1 X4/T-tropic IIIB and R5/M-tropic BaL. HIV-1 IIIB and BaL were incubated with rCCL20/MIP3α at concentrations of 2, 20, and 200 ng/mL for 1 hour at 37° C. The virus-MIP3α mixture was added to the TZM cells and incubated for 48 hours at 37° C. At the end of the incubation period, BETA-GLO substrate was added to the cells and viral infection was quantified as relative light units (RLU) using a luminometer. This analysis indicated that rCCL20/MIP3α inhibited both X4/IIIB and R5/BaL in multiple doses that were in the same range of rCCL20/MIP3α measured in endometrium and Fallopian tube secretions. Specifically, it was observed that constitutive CCL20/MIP3α secreted by epithelial cells ranged between 0.2-7 ng/mL in endometrium and 5-12 ng/mL in the Fallopian tube. When stimulated with Poly(I:C), the levels increased to 5-12 ng/mL by endometrium cells and 13-20 ng/mL by the Fallopian tube cells. The inhibitory activity was observed as a result of pre-incubation of HIV-1 and rCCL20/MIP3α, which indicates that the effect of CCL20/MIP3α on viral replication was likely to be direct.

Anti-HIV factors have been shown to inhibit HIV by multiple mechanisms, including direct interaction with HIV, by blocking cell surface receptors (CXCR4, CCR5), and by affecting post-infection steps. For example, macrophage inflammatory protein 1 alpha (MIP1α), beta (MIP1β), and Regulated on Activation, Normal T Expressed and Secreted (RANTES) are natural ligands for CCR5 that can therefore block entry of HIV into the cell. In addition, these molecules also function at a post-infection level by decreasing levels of intracellular cyclic AMP (cAMP) which is required for early steps in viral replication. Multiple mechanisms of HIV inhibition has also been observed for the well-characterized anti-HIV molecule SLPI.

After observing direct inhibitory effects of CCL20/MIP3α on HIV-1 infection, it was determined whether CCL20/MIP3α might also have indirect effects on viral infection. To determine whether CCL20/MIP3α acts by blocking HIV-1 receptors or co-receptors, TZM cells were pre-incubated with increasing concentrations of rCCL20/MIP3α for 1 hour at 37° C. After incubation, cells were washed with media to remove CCL20/MIP3α prior to the addition of HIV-1 IIIB and BaL viruses. Unlike what was observed with CCL20/MIP3α pre-incubation with HIV-1, no significant inhibition of either the X4/IIIB or the R5/BaL viruses was observed.

To determine whether CCL20/MIP3α acts through post-infection mechanisms, TZM cells were infected with IIIB and BaL, washed at 6 and 24 hours post-infection, after which CCL20/MIP3α was added to TZM cells. Other than a slight inhibition observed at 6 hours post-infection with the BaL virus, no significant post-infection inhibition was observed. In combination, these data indicate that the inhibitory activity of CCL20/MIP3α is likely through a direct interaction with the virus rather than at the level of the cell surface, or through disabling a later post-infection step.

Because these experiments indicated that antiviral activity might be due to epithelial cell production of CCL20/MIP3α, studies were undertaken to remove CCL20/MIP3α by antibody neutralization. To ensure that the antibody used was sufficient to remove CCL20/MIP3α, known amounts of rCCL20/MIP3α were first neutralized. It was found that neutralization of rCCL20/MIP3α (20 ng/mL) was possible with complete reversal of anti-HIV-1 activity. However, the anti-HIV-1 activity in secretions containing CCL20/MIP3α (<2 ng/mL) could not be reversed by using the same neutralization antibody at the same concentrations. It has been shown that FRT secretions contain a family of antimicrobials, some of which have anti-HIV-1 activity. As CCL20/MIP3α is one of the many molecules present in these secretions, neutralization would not be measurable as a separate entity.

### Example 9

### Intrinsic Anti-HIV Activity in Cervical-Vaginal Secretions from HIV-Positive and HIV-Negative Women

Materials and Methods. Specimen collection and patient information were provided as a part of an observational study on HIV shedding in women. With informed consent, 32 HIV(+), sexually abstinent women (48 hour prior to sampling) between the ages of 16 and 41 years were recruited. Enrollment criteria included a normal menstrual history, not on hormonal contraceptives, CD4 T cell counts above 350 cells/mm³, and no exposure to anti-retroviral (ARV) drugs. Participants agreed to undergo colposcopic assessment, and were excluded for pregnancy, breastfeeding, menopause, or inter-menstrual bleeding in the previous three months. Additionally, women were excluded if they had douched, used any vaginal products, or had sexual intercourse during the 48 hours prior to CVL collection. CVL was collected by gently washing the cervicovaginal area with 10 ml of sterile normal saline (pH˜7.2). Following CVL collection, samples were centrifuged at 10,000×g for 5 minutes after which supernatants and cell pellets were stored at −80° C. until used. Women were tested for lower genital tract infections including but not limited to bacterial vaginosis (BV), Trichonomas vaginalis, Neisseria gonorrhea and Candida albicans. Race and ethnicity were self-defined by the women involved in the study. Analysis of plasma viral load (PVL) and genital tract viral load (GTVL) RNA as well as CD4 counts, age and race are shown in Table 8.

TABLE 8

| Patient # | RACE | AGE | PVL | GTVL | CD4 Count |
|---|---|---|---|---|---|
| 1 | B | 33 | <400 | <400 | 419 |
| 2 | B | 23 | 23000 | <400 | 459 |
| 3 | B | 35 | 3300 | <400 | 510 |
| 4 | B | 41 | 12000 | <400 | 517 |
| 5 | B | 38 | 20000 | 9700 | 580 |
| 6 | B | 27 | 32000 | <400 | 583 |
| 7 | B | 39 | 67000 | <400 | 628 |
| 8 | B | 30 | 1500 | <400 | 637 |
| 9 | B | 31 | <400 | <400 | 647 |
| 10 | B | 33 | 17000 | <400 | 685 |
| 11 | B | 27 | 6629 | <400 | 772 |
| 12 | B | 24 | 10000 | <400 | 863 |
| 13 | H | 31 | 840 | <400 | 426 |
| 14 | H | 27 | 9300 | <400 | 510 |
| 15 | H | 36 | <400 | <400 | 620 |
| 16 | H | 28 | <400 | <400 | 799 |
| 17 | H | 32 | 54000 | <400 | 813 |
| 18 | H | 31 | <400 | <400 | 899 |
| 19 | H | 27 | 3600 | 460000 | 901 |
| 20 | H | 16 | 5200 | 4700 | 1517 |
| 21 | W | 28 | 3500 | <400 | 496 |
| 22 | W | 22 | 2100 | <400 | 502 |
| 23 | W | 29 | 110000 | 300000 | 605 |
| 24 | W | 26 | 3400 | <400 | 631 |
| 25 | W | 18 | 8000 | <400 | 637 |
| 26 | W | 32 | <400 | <400 | 651 |
| 27 | W | 30 | <400 | <400 | 677 |
| 28 | W | 22 | 7600 | <400 | 822 |
| 29 | W | 29 | 870 | 38000 | 872 |
| 30 | W | 19 | <400 | <400 | 921 |
| 31 | W | 25 | <400 | <400 | 931 |
| 32 | W | 32 | 1800 | <400 | 1277 |

^{a}CVL were recovered from HIV(+) women and stored in the HERS repository. The lower level of detection of the viral load assay was 400 copies/mL. Women self-identified by race as Black (B), Hispanic (H) or White (W).

CVL from participants in this study were obtained from the Rhode Island site of the HIV Epidemiology Research (HER) study. Women ranged in age from 24 to 34 years. CVL was collected by gently washing the cervicovaginal area with 10 ml of sterile normal saline (pH˜7.2). Following CVL collection, samples were immediately frozen at −80° C. as were HIV(+) samples. At the time of analysis, samples were thawed to room temperature, centrifuged at 10,000×g for 5 minutes after which supernatants were assayed for anti-HIV activity. Race was self-defined by the women involved in the study.

Laboratory-adapted viral strains HIV-1 IIIB (×4) and BaL (R5) were obtained and virus stocks were propagated in PHA-stimulated human PBMC and stored frozen at −80° C. Virus stocks produced in PBMC of molecularly cloned HIV-1 NL4.3 (×4; lab-adapted) and YU-2.c (R5; directly cloned without culture) were obtained. Virus titers were determined on TZM-bl cells as described herein. Also used was a PBMC-derived virus stock of pCH077.c, a recently generated Clade B infectious molecular clone matching the nucleotide sequence determined to be the transmitted/founder virus sequence of CHAVI subject 700010077.

Intrinsic anti-HIV activity in CVL was determined with TZM-bl cells. To measure the presence of infectious HIV-1 in CVL, TZM-bl cells were seeded at 2×10⁴ cells per well in a 96-well microtiter plate and allowed to adhere overnight at 37° C. Patient CVL samples were diluted 1:4 in TZM-bl media prior to 100 μl being added to TZM-bl cells for 48 hours at 37° C. in 5% CO₂. Luciferase activity was measured following manufacturer's instructions. All infectivity assays were performed in quadruplicate. Media diluted CVL had neutral pH values (pH 7.0-7.2).

Measurement of CVL anti-HIV-1 activity was determined as follows. TZM-bl cells were seeded at 2×10⁴ cells per well in a 96-well microtiter plate and allowed to adhere overnight at 37° C. CVL from individual patients were diluted 1:4 and incubated with virus (MOI=1) for 1 hour at 37° C. in a final volume of 100 μl. Following incubation, media was aspirated from TZM-bl cells and the virus plus CVL mixture (100 μl) was added to the cells along with 100 μl of TZM-bl media. Luciferase activity was measured as described herein. Controls included incubation of TZM-bl with virus alone, CVL alone and cells in media. Uninfected cells were used to determine background luminescence and data was expressed in relative light units (RLU). To calculate percent inhibition, the RLU values of “virus only” wells were averaged and set to 100%. Values of CVL treated virus were calculated as a percentage of the “virus only” value. Samples with values>100% were rounded to 100 and samples with <0% were rounded to 0. Viability of TZM-bl cells upon treatment with CVL was quantified using the CELLTITER 96® AQueous One Solution Cell Proliferation Assay (Promega) according to manufacturer's instructions. Briefly, reagent was added directly to cell cultures and incubated for 1 hr at 37° C. followed by reading the plate in a plate reader at OD 490 nm.

CVL supernatants were stored at −80° C. until assayed for SLPI, MIP3α and trappin-2/elafin with ELISA test kits or ELISA Duoset kit from R&D Systems (Minneapolis, Minn.) according to the manufacturer's protocol. Standards for each ELISA were re-suspended in phosphate-buffered saline. Cytokines were quantified based on standard curves obtained using an ELISA reader (Dynex, Chantilly, Va.). HBD2 was assayed with an ELISA test kit from PeproTech (Rocky Hill N.J.).

Measurement of anti-gp160HIV IgG antibody levels in CVL was by kinetic ELISA (kELISA) adapted from an assay previously described for influenza (Wright, et al. (2002) AIDS Res. Hum. Retroviruses 18(17):1291-1300). The kELISA measures substrate activation every 9 seconds over the first 5 minutes of the enzymatic assay and plots the change in color per minute as mOD/minute using a THERMOMAX microplate reader (Molecular Devices, Sunnyvale, Calif.). Duplicate wells were coated with MN strain gp160 (Protein Sciences, Meriden, Conn.). An uncoated well was run with each sample to determine background, which was subtracted from the result. A standard curve with a known positive serum specimen was included in each assay as a measure of sensitivity and reproducibility. Biotinylated IgG (Bioscience International, Camarillo, Calif.) goat anti-human conjugate was used with streptavidin-HRP and ABTS.

Nucleic acid sequence-based amplification (BioMerieux, Durham, N.C.) was used to measure HIV-1 RNA (Cu-Uvin, et al. (2006) J. Acquir. Immune Defic. Syndr. 42(5):584-587). All results are expressed as copies per milliliter with a lower limit of detection of 2.6 log₁₀ (400) copies/ml for both plasma and CVL.

Data from both HIV infectivity assays and ELISA were analyzed by GraphPad Prism software (La Jolla, Calif.). Mean and standard error were calculated for all data sets. Comparisons of anti-HIV activity between Control and CVL groups were done using two-tailed unpaired t-tests. ELISA data between multiple groups were done using one-way ANOVA tests followed by Tukey post-tests if significance was observed. A P-value of less than 0.05 was taken as indicative of statistical significance. A generalized linear model was used to analyze the relationship between anti-HIV activity (measured as percent inhibition) and the amount of HIV specific IgG. Quasibinomial family and logit link was used in the generalized linear regression. The null hypothesis that anti-HIV activity was not associated with IgG level was tested. P-value of less than 0.05 was taken as indication of statistical significance for the association between IgG concentration and anti-HIV activity.

Results. Table 8 shows the patient profiles of HIV(+) women indicating age, race/ethnicity, PVL, GTVL as well as CD4 counts. In these clinics, HIV viral load was measured by PCR and defined as viral RNA copies per ml. This measurement, however, does not distinguish between infectious and noninfectious HIV. To date, the issue of whether there is a correlation between RNA viral load in CVL and the presence of infectious virus has not been resolved. With this in mind, HIV infectivity was measured to determine the relationship between GTVL, PVL and the presence of infectious virus in the HIV(+) patient population. CVL from 32 HIV-infected women were evaluated for infectious virus. CVL were obtained from HIV(+) women who had CD4+ cell counts greater than 350 cells/mm³ and were not on ARV therapy. Samples were diluted 1:4 in media (final pH 7.0-7.2) and added to TZM-bl cells to assess whether virus present in CVL was infectious. This analysis indicated that approximately 10% (3/32) of CVL contained virus capable of infecting TZM-bl cells as measured by luciferase readout. No correlation was observed between GTVL (Table 8), PVL, the presence of STD or bacterial vaginosis, and infectious virus in CVL.

To determine whether CVL from HIV(+) women had direct inhibitory activity against HIV-1, individual specimens were incubated with either X4- or R5-tropic HIV-1 at a MOI=1 for 1 hour at 37° C. prior to the measurement of HIV-1 infectivity via the TZM-bl assay. A spectrum of anti-HIV activity was found ranging from 0 to 100% inhibition in CVL against both IIIB (×4) and BaL (R5) viruses. When compared to virus alone, the mean anti-HIV activity was significantly higher against IIIB than BaL, while the range of inhibitory activity was similar. Similarly, when anti-HIV activity was evaluated using 2 other viruses (NL4.3 and YU-2), significant anti-viral activity was observed against NL4.3 (×4) with limited, though not significant, inhibition (p=0.17) with YU-2.c (R5). When mean % inhibition±SEM of X4/IIIB (59±6.3%) was compared to X4/NL4.3 (59±6.6%) and R5/BaL (40±6.1%) was compared to R5/YU-2.c (33±5.5%), no appreciable differences were observed. Despite the limited number of viruses tested, the results indicate a greater inhibitory activity against the X4 viruses (59 and 59%) than that seen with R5 viruses (40 and 33%).

To evaluate whether CVL from HIV(−) women have endogenous anti-HIV activity, samples from 15 HIV(−) women were obtained from the HER Repository. Similar to that seen with HIV(+) CVL, some CVL samples had potent anti-HIV activity while others had little to no anti-viral activity. As with the CVL from HIV(+) women, HIV(−) CVL significantly inhibited IIIB and BaL as well as NL4.3 and YU-2.c virus infectivity of TZM-bl cells. To determine whether anti-HIV activity varies with race, the CVL data from HIV(−) women was examined based on racial self-identification. This analysis indicated that whereas each group (Black, White, and Hispanic) had anti-HIV activity in their CVL, no evidence of racial differences was measured. In contrast, HIV(+) CVL from Hispanic women had significantly (p<0.03) greater anti-HIV activity against BaL and CHO77.c (R5) viruses (60±13% and 64±10.5% inhibition; mean±SE, respectively), but not X4 viruses, than did CVL from Black women (26.4±7.5% and 32±7.5% inhibition). Despite this difference, what was clear from these results was measurable anti-HIV activity in CVL from HIV(+) and HIV(−) women irrespective of whether they were White, Hispanic or Black.

With the availability of infectious molecular clones containing the complete nucleotide sequences of transmitted/founder genomes, the susceptiblity of this virus to endogenous microbicides present in CVL was examined. This analysis indicated that this R5 virus was inhibited by CVL from HIV(+) and HIV(−) women. These findings indicate that the CHO77.c transmitted/founder virus is as susceptible to inhibition by FRT endogenous antivirals as the other strains tested.

To begin to define the mechanism(s) through which CVL from HIV(+) and (−) women exert their anti-HIV activity, secretion of specific key molecules were measured. Analysis of CVL from HIV(+) and (−) women for SLPI, trappin-2/elafin, MIP3α, and HBD2 indicated that each of these antivirals were present in CVL irrespective of race and HIV status. Recognizing that CVL specimens from HIV(+) and (−) women were obtained at different times, from different repositories with slightly different protocols, it was unexpected to observe that concentrations of MIP3α and SLPI were virtually identical. Moreover, the concentrations of SLPI in CVL in these studies were comparable to that reported previously by others (Keller, et al. (2007) AIDS 21(4):467-476). Unexpectedly, it was found that HBD2 levels were approximately 3-fold greater in HIV(+) women than that seen in HIV(−) women. Irrespective of these changes, however, neither the levels of antivirals nor total protein measured in CVL correlated with anti-HIV activity in these samples. Since as many as 12-20 antivirals are known to be present in CVL, these findings indicate that antiviral activity may be due to other individual molecules or the combined actions of several antimicrobials, including those analyzed here, which act either additively or synergistically to confer protection as reported previously.

To determine whether the CVL of HIV(+) women had antibodies against HIV, 15mOD was used as a cut-off for detection (Wright, et al. (2002) AIDS Res. Hum. Retroviruses 18(17):1291-1300) to assess 32 CVL samples for the presence of anti-HIV specific IgG and IgA antibodies. Thirty out of 32 CVL from HIV(+) women had anti-gp160HIV IgG antibodies, none were positive for anti-HIV IgA antibodies. As a control, CVL from HIV(−) women were tested and shown to lack IgG and IgA HIV-specific antibodies. To determine whether the presence of anti-gp160HIV IgG antibodies correlated with the anti-HIV activity, antibody levels were compared with anti-HIV activity in CVL. When plotted against inhibition of X4 virus NL4.3 and IIIB as well as R5 BaL and YU-2.c, a positive correlation between HIV inhibition and HIV-specific IgG antibodies in CVL was found. In addition, a positive correlation was found between inhibitory activity against CHO77.c (transmitted/founder virus) and concentration of HIV specific IgG antibodies. The wide width of the prediction intervals reflected the heterogeneity of the samples, suggesting that factors in addition to IgG antibodies were contributing to anti-HIV activity. This was reinforced by the observation that HIV(−) CVL had anti-HIV activity that was approximately the same as that seen in CVL from HIV(+) women.

### Example 10

### Broad Spectrum Endogenous Antimicrobials

Epithelial cells were isolated from the uterus, Fallopian tube and cervix tissues obtained from pre-menopausal and post-menopausal hysterectomy and grown in inserts until polarized to mimic in vivo conditions. Apical conditioned media was collected at 48 hours, and incubated with either Neisseria gonorrhoeae, Candida albicans or HIV-1 (X4/T-tropic and R5/M-tropic) for 1-2 hours prior to quantification of pathogen replication. Conditioned medium from FRT epithelial cells incubated with Lactobacillus crispatus was also tested to determine whether conditioned medium was inhibitory to commensals found in the lower FRT.

When apical secretions from uterus epithelial cells were incubated with known pathogens, conditioned medium was effective in inhibiting N. gonorrhoeae, C. albicans and HIV-1 (×4 and R5). Moreover, it was observed that medium from epithelial cells of the Fallopian tube and cervix also contained anti-microbial agents that inhibited N. gonorrhoeae and C. albicans. In contrast, conditioned medium from epithelial cells from all 3 sites failed to inhibit L. crispatus.

In so far as uterine epithelial cell secretions derived from patients in the proliferative and secretory phases of the menstrual cycle can inhibit S. aureus viability by 90% (Fahey, et al. (2002) J. Infect. Dis. 185:1606-13), it is contemplated that efficacious microbicides can be isolated from cells at these particular phases of the menstrual cycle.

These findings demonstrate the critical role of upper and lower FRT mucosal epithelial cells in providing innate immune protection against STI, without affecting commensal organisms. Therefore, anti-microbial agents can be isolated by conventional methods (e.g., column chromatography), characterized, and used in the prevention of sexually transmitted infections.

1. A method for boosting innate immunity comprising contacting cells of the female reproductive tract with a TGF-beta inhibitor thereby boosting innate immunity.

2. A method for preventing a sexually transmitted infection comprising locally administering an effective amount of a TGF-beta inhibitor to the reproductive tract of a female subject thereby preventing a sexually transmitted infection in the female subject.

3. The method of claim 2, wherein the female subject is pre-menopausal or post-menopausal.

4. The method of claim 2, wherein the sexually transmitted infection is human immunodeficiency virus.

5. The method of claim 1, wherein the TGF-beta inhibitor is a neutralizing antibody specific for a human TGF-beta, a soluble TGF-beta receptor, a membrane-bound TGF-beta receptor, a protease inhibitor that inactivates a protease responsible for activating a precursor TGF-beta into mature TGF-beta, an antibody specific to a TGF-beta receptor, siRNA to TGF-beta, antisense RNA to TGF-beta, latency-associated protein, decorin, or combinations thereof.

6. A method for boosting innate immunity comprising contacting cells of the female reproductive tract with a Selective Estrogen Receptor Modulator or Estrogen Receptor Antagonist thereby boosting innate immunity.

7. A method for preventing a sexually transmitted infection comprising locally administering an effective amount of a Selective Estrogen Receptor Modulator or Estrogen Receptor Antagonist to the reproductive tract of a female subject thereby preventing a sexually transmitted infection in the female subject.

8. The method of claim 7, wherein the female subject is pre-menopausal or post-menopausal.

9. The method of claim 7, wherein the sexually transmitted infection is human immunodeficiency virus.

10. The method of claim 6, wherein the Selective Estrogen Receptor Modulator is Tamoxifen, Droloxifene, Toremifene, Idoxifene, Clomiphene, Enclomiphene, Zuclomiphene, Raloxifene, Y134, LY 353381, EM 800, EM 652, Lasofoxifene, Levormeloxifene, bazedoxifene acetate, PHTPP, or analogs, derivatives, isomers, metabolites or mixtures thereof.

11. A method for boosting innate or adaptive immunity comprising contacting cells of the female reproductive tract with a low-dose glucocorticoid thereby boosting innate or adaptive immunity.

12. A method for preventing a sexually transmitted infection comprising administering an effective low-dose amount of a glucocorticoid to the reproductive tract of a female subject thereby preventing a sexually transmitted infection in the female subject.

13. The method of claim 12, wherein the dose is 0.5 to 5 μg/kg/minute.

14. The method of claim 12, wherein the sexually-transmitted infection is human immunodeficiency virus.

15. The method of claim 11, wherein the glucocorticoid is prednisone, prednisolone, methylprednisolone, dexamethasone, cortisol, hydrocortisone or combinations thereof.

16. A method for preventing a sexually transmitted infection comprising administering to a subject in need of treatment a recombinant commensal bacterium which expresses an endogenous microbicide to the intestinal tract or reproductive tract of a subject.

17. The method of claim 16, wherein the commensal bacterium is Lactobacillus sp., Staphylococcus epidermidis, Escherichia coli, Bifidobacterium bifidum, Neisseria sp. or Streptococcus grordonii.

18. The method of claim 17, wherein Lactobacillus sp. is L. jensenii, L. acidophilus, L. fermentum or L. crispatus.

19. The method of claim 16, wherein the endogenous microbicide is SLPI, HBD2, MIP3a, a CD4 protein, or a defensin.

20. The method of claim 16, wherein the bacterium selectively expresses the microbicide at least during mid-menstrual cycle.

21. The method of claim 20, wherein the microbicide is selectively expressed under the control of a hormone-responsive element.

22. The method of claim 2, wherein the TGF-beta inhibitor is a neutralizing antibody specific for a human TGF-beta, a soluble TGF-beta receptor, a membrane-bound TGF-beta receptor, a protease inhibitor that inactivates a protease responsible for activating a precursor TGF-beta into mature TGF-beta, an antibody specific to a TGF-beta receptor, siRNA to TGF-beta, antisense RNA to TGF-beta, latency-associated protein, decorin, or combinations thereof.

23. The method of claim 7, wherein the Selective Estrogen Receptor Modulator is Tamoxifen, Droloxifene, Toremifene, Idoxifene, Clomiphene, Enclomiphene, Zuclomiphene, Raloxifene, Y134, LY 353381, EM 800, EM 652, Lasofoxifene, Levormeloxifene, bazedoxifene acetate, PHTPP, or analogs, derivatives, isomers, metabolites or mixtures thereof.

24. The method of claim 12, wherein the glucocorticoid is prednisone, prednisolone, methylprednisolone, dexamethasone, cortisol, hydrocortisone or combinations thereof.
